(19) United States
(12) Patent Application Publication
(10) Pub. No.: US 2022/0040615 A1
(43) Pub. Date: Feb. 10, 2022

(54) FACE MASK

(71) Applicant: Christopher H. Cooper, Santa Fe, NM (US)

(72) Inventor: Christopher H. Cooper, Santa Fe, NM (US)

(21) Appl. No.: 17/392,946

(22) Filed: Aug. 3, 2021

Related U.S. Application Data
(60) Provisional application No. 63/060,952, filed on Aug. 4, 2020.

(51) Int. Cl. B01D 39/20; A62B 23/02; A62B 18/08; A61L 9/16; B01D 39/16; B01D 53/86; A41D 13/11

(52) U.S. Cl. CPC B01D 39/2065; B01D 2239/0216; A62B 18/084; A61L 9/16; B01D 39/1607; B01D 53/8668; A41D 13/11; A61L 2209/16; A61L 2209/14; A61L 2209/15; A61L 2209/111; B01D 2239/0241; B01D 2239/0442; B01D 2255/20; B01D 2257/708; A62B 23/025

(57) ABSTRACT

A face mask containing a network of conductive activated carbon meso-fibers crosslinked with a crosslinking agent bonded to a insulative polymeric micro-fiber based purification element. The purification element is captured between two ridged porous elements and contacted to the face by a biocompatible flexible polymeric seal. Optionally the front cover attaches to a main frame of the mask through a system of sliding locks that are coupled with bayonet latch mechanisms. The face mask attaches to the head of a wearer through a harness that couples with the sliding locks or alternatively with a biocompatible adhesive.

### BACKGROUND

### 1. Field

The presently disclosed instrumentalities relate to the field of face masks and half face respirators that remove particles or chemicals from air and, particularly, to half-face masks having filtration cartridges or other media that may be replaced or rejuvenated from time to time.

### 2. Statement of The Problem

A variety of face masks are known of the art. These masks include simple cloth bandanas that may be wrapped with rubber bands which loop around the ears, as well as mass produced masks that have renewable cartridges that are specially constructed for size exclusion of airborne particles and/or chemisorption of undesirable chemicals from the air. U.S. Pat. No. 6,732,733 provides one example of a half mask respirator utilizing specialized porous filtering mask bodies.

Modern carbon fibers are typically made by carbonizing fibers of rayon, pitch or polyacrylonitrile. The heat of carbonization may be, for example, around 1000° C. which is sufficient to produce a substance that is about 99 percent carbon having a poorly ordered graphitic lattice. Further heating to about 2,500° C. converts the material to 100 percent carbon and may also improve the ordering of the lattice. Pitch is an especially preferred starting material. Carbon nano-fibers and meso-fibers may be purchased on commercial order from such companies as Cytec Carbon Fibers, LLC of Piedmont, S.C. See High Performance Carbon Fibers, National Historic Chemical Landmark Commemorative Handbook, American Chemical Society, Sep. 17, 2003, which is incorporated by reference to the same extent as though fully replicated herein. These carbon fibers may be activated, for example, by exposure to carbon dioxide at temperatures greater than 850° C. or steam at temperatures greater than about 700° C. See Lee et al, Activated Carbon Fiber—The Hybrid of Carbon Fiber and Activated Carbon, Rev. Adv. Matter Sci 36 (2014) 118-136, which is incorporated by reference to the same extent as though fully replicated herein. For one skilled in the art of electrospinning the fibers described above can be drawn down to 10 nm to 100 nm in diameter prior to carbonization.

Problems have long-existed in the use of face masks. People wearing masks are generally more difficult to understand because they are heard to be mumbling as the mask interferes with speech, either in restricting the movement of the lips or suppressing sound waves travelling from it. When tasked with high efficiency removal of particles, pathogens, or chemisorption, the cartridges become bulky and heavy. The harness attaching the face mask to the head must be uncomfortably stretched out and deformed as it is mounted on the head. Furthermore, masks leak air around the filter thus compromising effectiveness. Respirators on the other hand are made to seal on the face so there is no leakage but have an air exit valve thus creating a biohazard if the user is infected with an airborne contagion. The present disclosure relates to a respirator that purifies air bi-directionally and seals to the face, containing a high flow purification element accomplished through the utilization of activated carbon meso-fibers.

### Definitions

The following terms or phrases used in the present disclosure have the meanings outlined below:

The term “fiber” or any version thereof, is defined as a high aspect ratio material. Fibers used in the present disclosure may include materials comprised of one or many different compositions.

The term “activated carbon meso-fiber” refers to a fiber substantially comprised of activated carbon, especially carbon fibers with a cross sectional area between 80 nm to 120 nm and length between 0.8 mm and 1.2 mm.

The phrase “lattice distortion” means any distortion of the crystal lattice of activated carbon meso-fiber atoms forming the fiber structure. Non-limiting examples include any displacements of atoms because of inelastic deformation, or chemical interaction followed by change in sp2 hybridization of carbon atom bonds. Such defects or distortions may lead to a natural bend in the activated carbon meso-fiber.

The term “functional group” is defined as any atom or chemical group that provides a specific behavior. The term “functionalized” is defined as adding a functional group(s) to the surface of the meso-fibers and/or the additional fiber that may alter the properties of the meso-fiber, such as zeta potential.

The term “doped” is defined as the insertion or existence of atoms, other than carbon, in the meso-fiber crystal lattice.

The term “charged” is defined as the presence of non-compensated electrical charge, in or on the surface of the activated carbon meso-fibers or the additional fibers. Such a charge may be the result of two fibers in contact that have significantly different work functions such as a activated carbon meso-fiber and a polymer fiber

The term “irradiated” is defined as the bombardment of the meso-fibers, the fibers, or both with particles, ions, photons such as x-rays, or with electromagnetic energy such as microwaves, to levels sufficient to cause inelastic change to the crystal lattice of the meso-fiber, fibers, differential heating to cause local heating to weld the activated carbon meso-fiber for the fiber or any other combination.

The terms “fused,” “fusion,” or any version of the word “fuse” is defined as the bonding of meso-fibers, fibers, or combinations thereof, at their point or points of contact. For example, such bonding can be Carbon-Carbon chemical bonding including sp3 hybridization or chemical bonding of carbon to other atoms.

The terms “interlink,” “interlinked,” or any version of the word “link” is defined as the connecting of meso-fibers and/or other fibers into a larger structure through mechanical, electrical or chemical forces. For example, such connecting can be due to the creation of a large, intertwined, knot-like structure that resists separation.

The terms “weaved,” “woven” or any version of the word “weave” is defined as the interlacing of meso-fibers and/or other fibers into a larger-scale material.

The terms “meso-structured” and “meso-scale” refer to a material having a minimum dimension with a peak distribution size of 10⁻⁷ m, i.e., 1 meso-meter or larger. A meso-fiber material may, for example, have a peak distribution dimension of 50 meso meters or 100 meso meters. Relevant definitions are provided in The Physics and Chemistry of Materials, Joel I. Gersten and Frederick W. Smith, Wiley publishers, p 382-383, which is herein incorporated by reference.

The term “Bio-soluble dopants” refers to dopants that will make the carbon soluble in the human body.

The term “rapid surface heating” refers to a fast thermal process that bonds the activated carbon nano-fibers to the polymer substrate.

The term “carbon media” refers to a meso-structured material defined above, and that further is porous. For example, in one embodiment, a carbon media material is generally used as a purification media, and thus must be porous or permeable to the fluid it is intended to purify.

The terms “large” or “macro” alone or in combination with “scale” refers to materials that comprise a meso-structured material, as defined herein, that have been fabricated using the methods described herein to have at least two dimensions greater than 1 cm. Non-limiting examples of such macro-scale, meso-structured material is a sheet of meso-structured material that is 1 meter square or a roll of meso-structured material continuously fabricated to a length of at least 100 meters. Depending on the use, large or macro-scale is intended to mean larger than 10 cm, or 100 cm or even 10 meters, such as when used to define the size of material made via a batch process. When used to describe continuous or semi-continuous methods, large scale manufacturing can encompass the production of material having a length greater than a meter, such as greater than one meter and up to ten thousand meters long.

The term “continuous method” refers to a method in which the deposition substrate continuously moves during the process until the fabrication of the meso-structured material is finished.

The term “semi-continuous method” refers to a method in which the deposition substrate moves, in a stepwise fashion, during the fabrication process. Unlike the continuous process, the substrate can come to a stop during a semi-continuous method to allow a certain process to be performed, such as to allow multilayers to be deposited.

The term “batch method” refers to a method in which the deposition substrate is stationary throughout the method.

The term “macro-material”, is a material having the lengths described above, e.g., as made by “large scale” or “macro-scale” manufacturing process described above.

The phrase “selective deposition substrate” as used herein refers to a substrate that is substantially transparent to the carrier fluid and substantially opaque to the said activated carbon meso-fiber composite components. For example, a filtration material that allows water to flow through but does not allow the activated carbon meso-fiber components to pass would be considered a selective deposition substrate. Such a substrate may be a 1 mm thick polymer felt.

The phrase “active material” is defined as a material that is responsible for a particular activity, such as removing contaminants from the fluid, whether by physical, chemical, bio-chemical or catalytic means. Conversely, a “passive” material is defined as an inert type of material, such as one that does not exhibit chemical properties that contribute to the removal contaminants when used as a purification media.

The term “fluid” is intended to encompass liquids, gases or supercritical fluids.

The phrase “loaded carrier fluid,” refers to a carrier fluid that further comprises at least activated carbon meso-fibers, and the optional components described herein, such as glass fibers.

The term “contaminant(s)” means at least one unwanted or undesired element, molecule, organism or biologically active material such as a viral particle in the fluid.

The term “removing” (or any version thereof) means destroying, modifying, deactivation or separating contaminants using at least one of the following mechanisms: particle size exclusion, absorption, adsorption, chemical or biological interaction or reaction.

The phrase “chemical or biological interaction or reaction” is understood to mean an interaction with the contaminant through either chemical or biological processes that renders the contaminant incapable of causing harm. Examples of this are reduction, oxidation, chemical denaturing, physical damage to microorganisms, bio-molecules, ingestion, and encasement.

The term “particle size” is defined by a number distribution, e.g., by the number of particles having a particular size. The method is typically measured by microscopic techniques, such as by a calibrated optical microscope, by calibrated polystyrene beads, by calibrated scanning probe microscope scanning electron microscope, or optical near field microscope. Methods of measuring particles of the sizes described herein are taught in Walter C. McCrone's et al., The Particle Atlas, (An encyclopedia of techniques for small particle identification), Vol. I, Principles and Techniques, Ed. 2 (Ann Arbor Science Pub.), which are herein incorporated by reference.

The phrases “chosen from” or “selected from” as used herein refers to selection of individual components or the combination of two (or more) components. For example, the meso-structured material can comprise activated carbon meso-fibers that are only one of impregnated, activated functionalized, doped, charged, coated, and defective activated carbon meso-fibers, nano-carbon, carbon nanotubes or a mixture of any or all of these types of meso-fibers such as a mixture of different treatments applied to the meso-fibers.

The phrase “paper machine clothing” as used herein refers to a carrier substrate. The definition incorporates by reference what is described in Sabit Adanur et al., Paper Machine Clothing, second edition, (AstenJohnson).

The phrase “Nonlinear flow restriction membrane” refers to a micro sized porous membrane that forces a substantially uniform flow across when a pressure differential is applied. The NFRM can have an average pore size of between 1 um and 10 um. In the preferred embodiment the pore size is 2.7 um. The NFRM can be made from but not limited to polypropylene, stainless steel, or cellulose.

The term “Synthesis bi-products” is referred to as molecular components leftover from the synthesis of the conducting activated carbon meso-fibers. This material typically consists of metal catalysts such as iron and poly-aromatic hydrocarbons.

The term “Preferential electromagnetic radiation” refers to radiation that is preferentially adsorbed by the meso-fibers and converted to heat while poorly adsorbed by the polymer fiber.

The term “Nitric acid wash” refers to the following method for the removal of synthesis bi-products from the activated carbon meso-fibers. First the activated carbon meso-fibers are dispersed in a concentration of cold nitrogen packed 3 molar nitric acid. Second the acid is brought to 80 C and the solution is stirred for one hour with a magnetic stirring heating jacket. This reaction vessel is connected vertically to a Vernier or cooling column so vaporized water and acid fumes can condensed and drip back into the reaction vessel. The arrangement is kept under a slight pressure of nitrogen the “nitrogen jacket” Oxygen is kept from the reaction because otherwise it would react with and perform damage to the carbon comprising the meso-fiber. This reaction is maintained for one hr.

As used herein, the term “purification media” means a media that acts upon an incoming fluid, such as air or water, to remove unwanted materials from the fluid. The fluid may be, for example, air or water or a supercritical material, and the purification media may be by adsorption or chemisorption. Additional methods of action for purification may include, for example, a breakdown of material such as by catalysis of volatile organic materials or toxins in the case of biological pathogens, the destruction of viruses or bacteria by the death or deactivation thereof. A purification material may also operate by size exclusion of pathogens of other undesirable materials.

### SUMMARY

The presently disclosed instrumentalities overcome the problems outlined above and advance the art by providing a high efficiency face mask with replaceable cartridges or covers, the performance of which may be electronically enhanced.

In one aspect, the face mask may include a main frame element that is permeable to air. A bulbous flexible face guard is constructed to conform for sealing engagement with contours of a human face. The face guard is in sealing engagement at an interface with a rearward portion of the main frame to prevent leakage of air therebetween. A front cover element is also permeable to air. A space between the main frame and the front cover is fitted with at least one purification media element including conductive activated carbon meso-fibers supported by a network of micro polymeric fibers. A binding agent, such as a crosslinked polymer, connects the conductive activated carbon meso-fibers and the a network of micro polymeric fibers, the conductive activated carbon meso-fibers being present in an effective amount for the purification-removal of unwanted constituents from air.

In one aspect, the purification media may be provided with electronic enhancements. These enhancements may be, for example, circuitry establishing a potential difference across the filter media; static charging due to airflow through a purification media comprising conductive activated carbon nano-fibers (electron donors) and insulative (electron accepter) micro-fiber material, piezoelectric devices, capacitive devices, photo-voltaic cells, batteries, sensors, wires, digital circuitry, analogue circuitry or any combination thereof.

In various aspects of suitable materials, the main frame element may be made of thermal set polymer, epoxy, metal, ceramic, wood or any combination thereof. The polymeric micro-fiber may be, for example, selected from synthetic polymers, biopolymers, proteins, cellulose, wool, cotton or any combination thereof. The crosslinking agent may be, for example, selected from but not limited to: chitosan, DNA, RNA, synthetic polymers, biopolymers, or any combination thereof. The flexible face guard is suitably made of a soft biocompatible silicon polymer, polymer, bio-polymer or any combination thereof.

In one aspect, the face mask include a harness for retaining the face mask on the head of a wearer. Alternatively, the face mask may be retained on the head of a wearer by a layer of bio-compatible adhesive located between the flexible face guard and the face of the wearer. Where a harness is utilized, the harness may attach at sliding locks that are formed in two pieces and are coupled by a bayonet latch mechanism.

In various aspects, the purification media may contain, for example, at least one metal oxide catalyst for the removal of volatile organic compounds from the air. The purification media may be functionalized to facilitate the removal of biological pathogens from the air.

The purification media has additional uses beyond the purification media as described above. A standalone purification media may contain, for example, polymeric microfibers, conductive activated carbon meso-fibers, and a binding agent coupling the polymeric microfibers and the conductive activated carbon meso-fibers. The purification media is permeable to fluid flow, including gaseous fluids and/or liquid fluids. The activated carbon meso-fibers are present in effective amounts for the purification-removal of unwanted constituents from air. These constituents may include, for ex ample, viruses, bacteria, and volatile organic compounds. The purification media may be incorporated into conventionally sized filters of the type commonly used in such systems as HVAC systems, and systems for the recirculation of air in vehicles such as cars or airplanes.

In one aspect, the main frame presents a first plurality of rails, and the front cover presents a second plurality of rails. The first plurality of rails is placed into alignment with the second plurality of rails when the front cover is positioned over the main frame to form at least two rail groupings of first and second rails in alignment. At least two sliding locks are each respectively positioned over a selected one of the rail groupings to retain the front cover on the main frame, the sliding locks being alternatively positionable for selective removal and retention of the front cover on the main frame.

In one aspect, the face mask may include electronic enhancements mounted on the main frame. The electronic enhancements may include circuitry establishing a potential difference across the media. By way of example, the potential difference may be low, on the order of millivolts, to remove substantially all airborne viral particles in the intended environment of use, depending upon the type of virus.

In one aspect, the electronic enhancements may include biometric sensing of a condition of the wearer and may be transmitted to a network-based monitor.

In one aspect, the electronic enhancements may include a microphone and circuitry communicating voice communications detected though the microphone to an external speaker or circuitry routing the signal through nearfield communications.

Disclosed below is a method for making meso-structured materials comprising adsorption of carbon structures or meso-fibers onto at least one substrate fiber via a carbon suspension fluid contact station, wherein the suspension comprises transporting carbon components to the substrate from a deposition fluid, such as liquid or supercritical fluid. By using a substrate that is permeable to the carrier fluid, and allowing the carrier fluid to flow through the substrate by differential pressure filtration, a micro-meso or meso-structured carbon material can be formed on and in the substrate, which may be removed, or may act as a part of the final component.

In one aspect, this disclosure provides an efficient process for manufacturing large quantities of covalently bonded meso-composite material comprising activated carbon meso-fibers and other components, such as ceramic or polymetric fibers. The process is a continuous, semi-continuous or batch method of producing functional micro-meso-meso-structured material based on carrier fluid suspensions of carbon based structures contacting fibers, and welding said components with covalent bonding by particle bombardment.

Most two-dimensional materials, such as webs, sheets, and the like, have inherent shortcomings in their material properties. While metals and plastics have long been favorites because of their wide range of versatility, for many applications' higher strength-to-weight ratio, higher conductivity, larger surface area, higher tenability and overall higher performing materials are needed. Exotic lightweight, high strength materials used to be confined to high tech applications like space exploration and electronics, however, they are becoming increasingly important for mass applications in ballistic mitigation applications (such as bulletproof vests), heat sinks, fluid purification, fluid separation, high efficiency electrodes for batteries capacitors and fuel cells, computer casings, car bodies, aircraft wings, machine parts, and many other applications.

The field of nanotechnology as it relates to purification media has had for many years one major drawback, which is the extreme difficulty in preventing an unwanted release of nanoparticles into the purified fluid stream. The United States Environmental Protection Agency has very tight controls on any product that could result in harmful nano-particle release from a purification media. The present disclosure relates to the use of carbon based meso diameter fibers synthesized to 100's of microns, to millimeters in length as a base to prevent the release of nano-scale components. These meso-fibers can be fused to larger polymer fiber or polymer felt structures that smaller nanofibers or particle cannot. The method advantageously solves a major problem in the development and commercialization of these new classes of nano-structured purification media technologies and products.

Activated carbon meso fibers, activated carbon meso-fibers, or graphene clusters, have a wide-ranging conductivity, for example ranging from 1 picoamp/m² to 10¹⁷ amps/m², allows the material to be tailored for a wide variety of applications. Non-limiting examples of articles made from meso-structured material as described herein include fabrics, sheets, wires, structural supports, cables, tubes or membranes for fluid purification, elemental separation, transportation, and construction. Electrical, mechanical and thermal properties associated with the graphene-based composites further allow the meso-structured materials to be used for higher performance mechanical actuators, heat sinks, thermal conductors or electrodes.

Given the acute need for materials with these improved performance characteristics in many applications, there is a need for methods that produce these materials in large quantities. Accordingly, the present disclosure relates to a method of making a carbon or activated carbon-enabled material in large quantities, such that the resulting product can be sized for a variety of applications, from purification media to fabrics for electrical or mechanical uses.

In one embodiment, the process provides a method of making a meso-structured material comprising, for example, activated carbon meso-fibers 1 mm in length and 100 nm in diameter referred to herein as Activated carbon meso-Fibers (activated carbon meso-fiber). These activated carbon meso fibers may, for example, have a length distribution ranging from 0.8 mm to 1.2 mm centered on a mode at about 1 mm. The method typically comprises suspending activated carbon meso-fibers in a carrier fluid to form a mixture, inducing the mixture to flow through a substrate that is permeable to the carrier fluid by differential pressure filtration, and depositing the activated carbon meso-fibers (and optional components such as glass fibers), from the mixture onto the substrate. The large-scale meso-structured material is one having at least one dimension greater than 1 cm.

The present disclosure also relates to a continuous or semi-continuous method for making a meso-structured material comprising activated carbon meso-fibers. In this embodiment, the activated carbon meso-fibers are deposited from the mixture onto a moving substrate to form a meso-structured material having a length greater than 1 meter. This embodiment enables very large meso-structured material to be formed, such as a material having at least one dimension greater than 1 meter, for example a length of hundreds or thousands of meters, and up to ten thousand meters.

Also disclosed herein is disclosed a batch method for making a meso-structured material. Unlike the continuous or semi-continuous method, the batch method comprises depositing the activated carbon meso-fibers from a mixture onto a stationary substrate that is permeable to the carrier fluid. While a batch method does not typically permit materials to be formed of the same size, such as length, as the continuous or semi-continuous method, it is able to produce a macro-scale meso-structured material, such as one having at least one dimension greater than 10 cm.

The method described herein may be used to make a wide variety of novel products, such as a macro-scale, meso-structured material for purification of fluids. This method may be used to directly deposit a seamless meso-structured material onto a substrate that will become an integral part of the final product. In one embodiment, this method can be used to deposit macro-scale meso-structured material onto a purification media, such as a polymer based felt.

In one aspect, there is disclosed a method for making a meso-structured material from a carrier fluid based on a differential pressure technique. In one embodiment, the method comprises suspending activated carbon meso-fibers, and optionally other components, in a carrier fluid, depositing the activated carbon meso-fibers onto a substrate that is permeable to the carrier fluid, and allowing the carrier fluid to flow through the substrate by differential pressure filtration to form a meso-structured material. The method described herein can be used to produce large or macro-sized materials, such as a material having at least one dimension greater than 1 cm, or even greater than 100 cm.

The carrier substrate that may be used in the present disclosure may, for example, be comprised of fibrous or non-fibrous materials. Non-limiting examples of such fibrous and non-fibrous materials include metals, polymers, ceramic, natural fibers, and combinations thereof.

The carrier fluid described herein may include at least one aqueous and non-aqueous liquid, at least one gas, or combinations thereof. When used, the aqueous liquid may, for example, have a pH ranging from 5 to 8.9.

In one embodiment, the carrier fluid further comprises chemical binding agents, such as polyvinyl alcohol, urethane, poly-electrolytes, and combinations thereof. The carrier fluid may also or alternatively comprise biomaterials chosen from proteins, DNA, RNA, and combinations thereof.

The other components described herein may be pre-assembled and attached onto the activated carbon meso-fibers, to other components, or to any combination thereof prior to the deposition step.

In one embodiment, the method disclosed herein can be used to form a multilayered structured by the sequential deposition of at least one meso-structured layer and at least one additional layer, which may or may not be nano-structured, such as a layer of carbon nanotubes. In this embodiment the meso-fibers prevent the release of the carbon nanotubes in a purification device.

The method may further comprise the application of an acoustic field to obtain or maintain dispersion of the activated carbon meso-fibers in the carrier fluid prior to the depositing step. Non-limiting examples of an acoustic field that may be used in the disclosed method is one having a frequency ranging from 10 kHz to 50 kHz.

It is also possible to disperse and or mix the activated carbon meso-fibers in the carrier fluid by applying a high-shear flow field to the carrier fluid. This same process may be used to disperse and or mix the activated carbon meso-fibers with other components, when present.

It is also possible to use a combination of an acoustic field with the previously mentioned frequency range and a high-shear flow field, either sequentially or in combination, to obtain or maintain dispersion of the activated carbon meso-fibers in the carrier fluid prior to said depositing.

In various embodiments, the method further comprises treating the meso-structured material with at least one post-deposition treatment process. Non-limiting examples of such processes include (a) chemical treatment, such as adding a functional group, coating with another material (such as a polymer or metal) or both, (b) irradiation, such as exposing the meso-structured material to at least one radiation chosen from infrared radiation, optical radiation, ultra-violet radiation, electron-beams, ion beams, x-rays, photons, or any combinations of (a) and (b).

The post-deposition functionalization process described herein may comprise procedures chosen from: acid washing, surfactant treatments, molecular grafting, deposition of polyelectrolyte materials, coating, heating, spraying, chemical or electrolytic dipping, or combinations thereof.

The method described herein may further comprise finishing the meso-structured material to form a shape and size sufficient for a particular application. For example, finishing the meso-structured material comprises at least one method chosen from cutting, laminating, sealing, pressing, wrapping, or combinations thereof.

The disclosed method can be used in a continuous or semi-continuous fashion for making a meso-structured material. For example, the activated carbon meso-fibers are deposited via a carrier fluid, onto a moving substrate that is permeable to the carrier fluid. This embodiment enables very large meso-structured materials to be continuously formed, such as a material having at least one dimension greater than 1 meter, including length up to hundred and even thousands of meters.

There is also disclosed a batch method for making a meso-structured material. Unlike the continuous or semi-continuous method, the batch method comprises depositing the activated carbon meso-fibers onto a stationary substrate that is permeable to the carrier fluid. While a batch method does not typically permit materials to be formed of the same size, such as length, as the continuous or semi-continuous method, it is able to produce a macro-scale meso-structured material, such as one having at least one dimension greater than 10 cm.

The batch process for making a meso-structured material is particularly useful for producing a complex shape and/or a product that benefits from a seamless construction between the substrate and meso-structured material deposited thereon. In one embodiment, a purification media can be produced in which the underlying substrate forms an integral part of the purification, such as a polymer felt.

It has been discovered that the combination of activated carbon meso-fibers and glass fibers coated with metal-oxygen compounds provide exceptional purification properties when used to clean contaminated fluids. Thus, with regard to the use of the final product as a purification media, one embodiment described herein, it is believed that unlike activated carbon meso-fibers which serve as an active component, the primary role that the larger scale fibers, such as the glass fibers, serve as support for the active material(s). While the fiber may remove particulates from the fluid through a size exclusion principle, it typically is a passive, non-reactive element in the meso-structured material used to purification contaminated fluid.

Aside from the subject matter discussed above, the present disclosure includes a number of other exemplary features such as those explained hereinafter. It is to be understood that both the foregoing description and the following description teach by way of example and not by limitation.

### BRIEF DESCRIPTION OF THE DRAWINGS

FIG. 1 shows a face mask in the form of a half mask utilizing a system of sliding locks to couple various components of the mask;

FIG. 2 is an assembly view of the face mask showing components including a front cover, a main frame and an elastomeric face guard;

FIG. 3 is a front view of the cover;

FIG. 4 is a rear view of the cover showing a bound media for removing unwanted constituents of air;

FIG. 5 is a front view of the main frame;

FIG. 6 is a rear view of the main frame;

FIG. 7 is a front view of the face guard;

FIG. 8 is a rear view of the face guard;

FIG. 9 is a bottom view of the face guard;

FIG. 10 shows a female piece of a sliding lock assembly;

FIG. 11 shows a male piece of the sliding lock assembly;

FIG. 12 is an assembly view of a bayonet latch mechanism that couples the female and male pieces of the sliding lock assembly;

FIG. 13 is a front view of a second embodiment of the main frame that is improved by the addition of electronic enhancements;

FIG. 14 shows circuitry for electrostatic enhancement of a filtration media;

FIG. 15 is a circuitry schematic for electronic enhancement of the main frame by the addition of sensor array and associated circuitry;

FIG. 16 is a side perspective view showing the face mask on the head of a wearer;

FIG. 17 is a flow chart of a activated carbon meso fiber deposition process according to one embodiment.

FIG. 18 is a schematic of a system according to one embodiment for the batch production of a meso-structured material for use as a purification media according to the present disclosure; and

FIG. 19 is a schematic of a system according to one embodiment for the web-based production of a meso-structured material for use as a purification media according to the present disclosure.

### DETAILED DESCRIPTION

The following discussion teaches by way of example and not by limitation. Therefore, what is disclosed should not be used in an undue manner for purposes of unnecessarily limiting what is claimed.

### Mask Structure

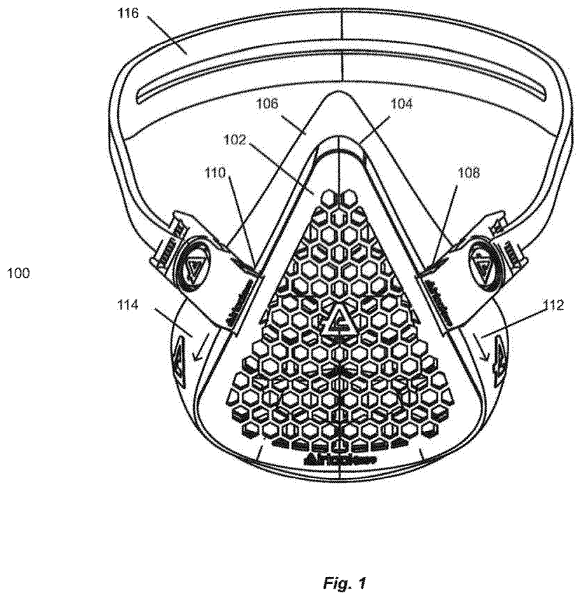

FIG. 1 shows an exemplary face mask assembly 100 according to one embodiment of the present disclosure. The face mask assembly 100 includes a front cover 102 that may be constructed as a cartridge that is bound a filtration, purification or chemisorption media 500 (see FIG. 4) as described below, or else the purification media 500 may be compressively retained between the front cover 102 and the main frame 104. The front cover 102 is detachably mounted on a main frame 104 that is over-molded with an elastomeric face guard 106 for support thereof. The face guard 106 may be made, for example, of a soft biocompatible silicon polymer, polymer, bio-polymer or any combination thereof. The face guard 106 is constructed to conform to a wearer's face (not shown) for sealing engagement therewith covering the nose and mouth of the wearer. The sealing engagement may be enhanced by use of an adhesive at the boundary between the face guard and the wearer's face. This boundary that is optionally coated with adhesive is shown, for example, as wall 900 in FIG. 8. A pair of sliding locks 108, 110 compress the front cover 102 and the main frame 104 into sealing engagement with one another. The sliding locks 108, 110 may be moved in the direction of arrows 112, 114 for selective positioning to engage the front cover 102 against the main frame 104, or to permit the front cover 102 to detach from the main frame 114. An elastomeric harness 116 has a first band that stretches around the back of a wearer's neck (not shown) and a second band that stretches around the back of a wearer's head for support of the face mask 100 on a wearer's face. The elastomeric harness 116 is optionally omitted when the adhesive is used as described above. The main frame 104 is suitably made, for example, from a material such as thermal set polymer, epoxy, metal, ceramic, wood, and combinations thereof. It will be appreciated that the front cover 102, main frame 105 and purification media 500 are all permeable to air and may be assembled as a unit permitting air to pass through the unit.

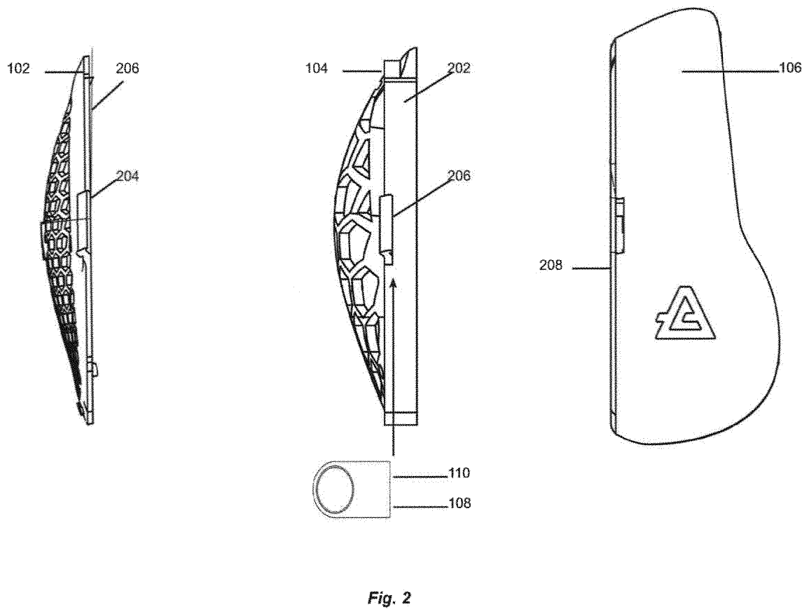

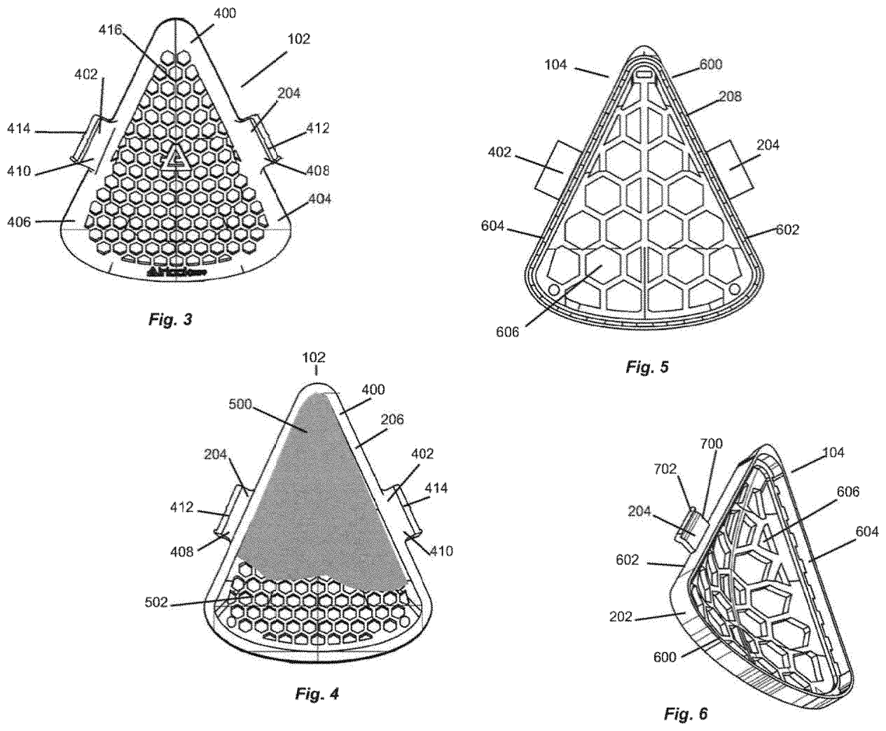

FIG. 2 is an assembly view of the face mask assembly 100. The face guard 200 is provided with a lip 200 that compressively overfits an outer rim 202 of the main frame 104 main frame 104 for sealing engagement therewith. The lip 200 is preferably over-molded to cover the outer rim 202 during manufacture. The front cover 102 and the main frame 104 are provided with rails 204, 206. When assembled, the rails 204, 206 are aligned in parallel for receipt of the sliding lock 110. A mirror-image alignment of rails (not shown) is provided for receipt of the sliding lock 108. The engagement with sliding locks 108, 110 compresses edge 206 of the front cover 102 against face 208 of the main frame 208 for sealing engagement therewith.

FIGS. 4 and 5 provide additional detail with respect to the front cover 102. A peripheral edge 400 is generally triangulated, tapering up. Forward rails 204, 402 are generally aligned with the center of mass in the front cover 102 and protrude at right angles from upwardly tapering side sections 404, 406. The rails 204, 402 may be integrally formed with the side sections 404, 406. The rails 204, 406 have necks 408, 410 that connect the respective side sections 404, 406 with heads 412, 414. The heads 412, 414 flare forwardly from necks 408, 410 and rises in parallel with the side sections 404, 406 for retention of the sliding locks 108, 110 (see FIG. 1). The peripheral edge 400 circumscribes a honeycomb wall 416. This is a wall with honeycomb openings for the passage of air therethrough and is generally concave out towards the front of the front cover 102.

FIG. 5 is a rear elevation view of the from cover 102 showing an internal media 500. The media 500 may optionally be any type of purification media, such as cloth or N95 capable size exclusion media as are known in the art. The media may also be, for example, a media that provides chemisorption such as by the action of activated carbon. As depicted in FIG. 5, the media 500 is over-molded by the peripheral edge for retention at a convex rear surface 502 of the honeycomb wall 416. Alternatively, the purification media 500 may be adhered to the peripheral edge 400. It will be appreciated that the purification media 500 covers the entirety of the rear surface 502 such that all air passing through openings in the honeycomb wall 416 passes through the purification media 500, and that FIG. 5 shows a lower portion of media 500 removed for purposes of illustration only to reveal the underlying rear surface 502 of the honeycomb wall 416.

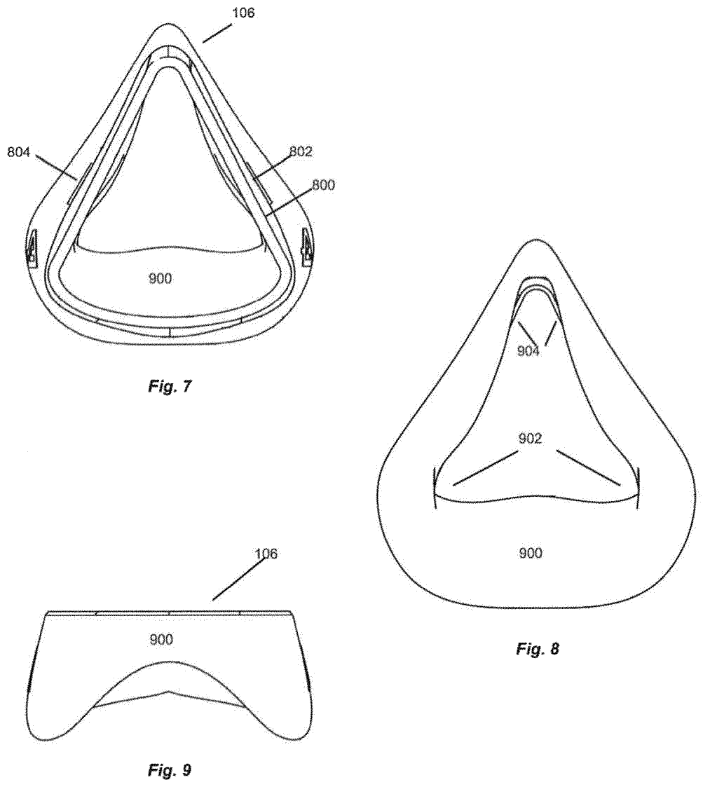

FIGS. 6 and 7 provide additional detail with respect to the main frame 104. Rearward 204′, 402′ align with forward rails 204, 302 of the front cover 102 (see FIG. 5) when the front cover 102 is operably placed over the main frame 104 for engagement with sliding locks 108, 110 (see FIG. 1). The main frame 104 has a peripheral edge 600 on which face 208 and surface 202 are formed. The peripheral edge 600 includes sides 602, 604 that triangulate up. The rails 204′, 402′ complement rails 204, 402 for engagement with the sliding locks 108, 110, for example, by the provision of neck 700 connecting side 602 with head 702. The head 702 flares rearwardly and rises in parallel with the side 602. The peripheral edge 600 circumscribes a honeycombed wall 606 that is concave forward and contains a plurality of openings for the passage of air therethrough.

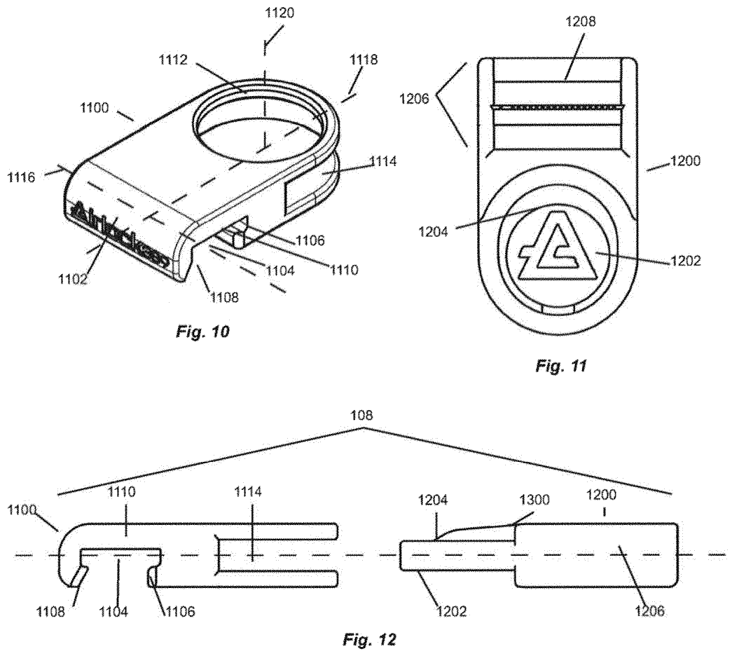

FIGS. 8, 9, and 10 provide additional detail with respect to the face guard 106. The front of the face guard 106 is molded to provide a pocket 800 that sealingly engages surface 202 of the main frame 104. Nibs 802, 804 extend outwardly from the exterior of the pocket 800 and align with rails 204′, 402′ to be overlain with the sliding locks 108, 110 (See FIG. 2) for engagement therewith to assist retention of the face guard 106 on the main frame 104. A bulbous hollow wall 900 conformably adapts to the face of a wearer for sealing engagement therewith to accommodate a wide range of face shapes and sizes (not shown). The bulbous wall 900 contains a lower portion 902 adapted to engage the chin area of a wearer, and tapers upward to a region that flexibly adapts to seal against the nose.

FIGS. 11, 12 and 13 provide additional detail with respect to the sliding lock 108, which is identical with respect to the sliding lock 110. In the embodiment shown, the sliding lock 108 is formed of two pieces including a female piece 1100 (FIG. 11) and a male piece 1200 (FIG. 12). The female piece 1100 is formed with a forward rounded lip 1102 extending to define the forwardmost limit of a longitudinally transverse slot 1104 with transversely extending tracks 1106, 1108. The tracks 1106, 1108 are separated by a distance such that the tracks 1106, 1108 form a compression fitting against heads 412 (FIG. 4), 702 (FIG. 7) when the front cover 102 is operably pressed against the main frame 104. A wall 1110 defines aperture 1112 and slot 114. These features are oriented such that an axis of symmetry 1116 in the slot 1104 runs perpendicular to an axis of elongation 1118 in the female piece 1100. An axis of symmetry 1120 in the aperture 1112 is perpendicular to both the axis of elongation 1118 and the axis of symmetry 1116.

The male piece 1200 is formed with a forward nose 1202 having complimentary dimensions for receipt within the slot 1114 and an integrally formed spring plate 1204. A rearward section 1206 contains a system of bars and slots that receive webbing from the harness 116 and permit adjustment of the length thereof.

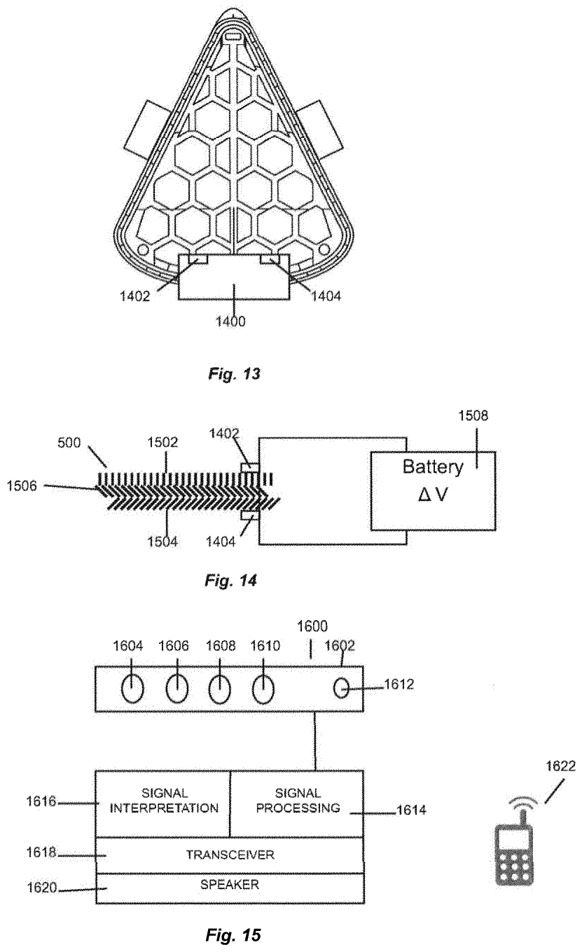

The female piece 1100 and male piece 1200 form a buckle that assembles as shown in FIG. 13. The forward nose 1202 is received within the slot 1114 such that the spring plate 1204 is depressed when entering the slot 1114 and springs back as a bayonet latch into a locking position against the aperture 1112 when a rearward most part 1300 is free to rise within the aperture 1112. Thus, the sliding lock 108 may be selectively detached by depressing the spring plate 1204 to disengage part 1300 from aperture 1112. It will be appreciated that his buckling arrangement by the union of male and female parts 1100, 1200 permits the harness 116 to be placed on the head of a wearer without having to stretch the harness out to accommodate the wearer's head.

### Electronic Enhancements

FIG. 14 shows a second embodiment in which a main frame 106′ is improved by the addition of electronic enhancements 1400. In one aspect the improvements may include electrostatic enhancement of the media 500 to improve the filtration efficiency thereof. As shown in FIG. 15, The electronic enhancements include circuitry 1500 such that contacts 1402, 1404 are placed in contact with the media 500. The contact 1402 is in electrical contact with a first conductive layer 1502 of media 500. The contact 1404 is in electrical contact with a second conducti9ve layer 1504 of the media 500. A relatively nonconductive layer 1506 is placed between the first and second conductive layers 1504, 1504 to prevent shorting of the voltage potential ΔV established by a battery 1508. A battery 1502 provides a potential difference. The first and second conductive layers 1502, 1504 may be, for example, activated carbon meso fibers, activated carbon meso-fibers or carbon nanotubes in a matrix of thermoplastic. All of the layers 1502-1506 permit the passage of air.

The conductive layers 1502, 1504 may be selectively arranged to configure the first conductive layer 1502 as a cathode or anode, depending upon whether the particles intended for filtration are positively or negatively charged. By way of example, viral capsids frequently have an argentine rich motif that presents a positive charge, so the first conductive layer 1502 could be negatively charged for electrostatic attraction and binding with these capsids when the first conductive layer is proximate to a flow 1510 of incoming air. Some viruses have positively charged tails. The circuitry could also be provided with pressure sensing circuitry that inverts the cathode and anode upon exhalation to diminish parasitic capacitance that might otherwise be caused by incoming particles. The potential may be reversed for negatively charged particles.

The voltage potential ΔV can be any level that is safe, but need not be large to have a significant effect toward removal of viral particles from the air. A potential difference on the order of millivolts may result in the removal of viral particles to achieve undetectable levels from media that are not accomplishing this result through size exclusion. Without being bound by theory, it is believed that the application of a small potential difference to activated carbon meso fibers, activated carbon meso-fibers or carbon nanotubes may result in deactivation of the virus, such as by rupture of the capsid or other phenomena.

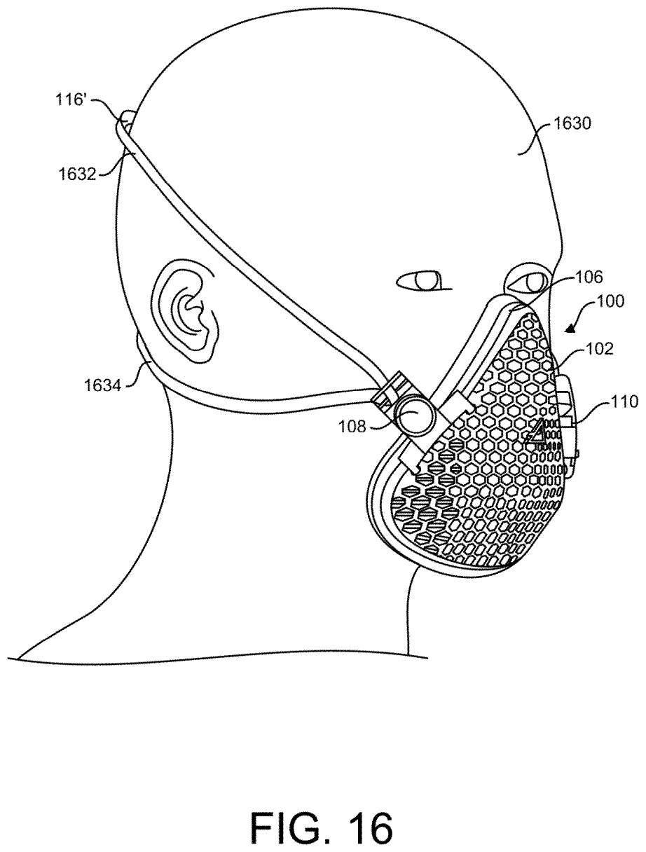

FIG. 16 shows another embodiment of the electronic enhancements 1400. Circuitry 1600 includes a sensor array 1602 that contains a plurality of sensors 1604, 1606, 1608, 1610 and a microphone 1612. Analog outputs from these sensors communicate with signal processing circuitry 1614 as needed for programmatic signal interpretation 1616. Signal processing circuitry 1614 includes signal filtering, amplification, and analog to digital conversion as are known in the art of signal processing. The programmatic signal interpretation utilizes, for example, correlations that are made for interpreting signals from the particular types of signals in use on the sensor array 1602.

Output flowing from the programmatic signal interpretation may result in near-field telecommunications through use of a transceiver 1618 using, for example, wireless nearfield communications to transmit and receive data from a network asset 1622 such as a smartphone or monitor 1622 forming part of the Internet of Things. This data transmission may, for example, result from an analysis of breath from the wearer of face mask 100 where the sensed signals are interpreted as showing a condition that should result in a warning. This could be inebriation from alcohol, an indication of a diabetic condition, a metabolite indicating an undesirable level of chemical exposure, or the presence of viral or other particles inside the face mask 100. If the signal is from microphone 1612, then voice communications may be programmatically routed through speaker 1620 or network asset 1622 in the form of a smartphone or other handheld telecommunications device.

The exact circuitry and signal processing logic for each sensor in the sensor array 1602 may vary by methods that are known in the art. For discussion of sensor circuitry and program logic used in sensors and sensor arrays, see U.S. Pat. No. 7,232,510 to Miyazaki et al.; U.S. Pat. No. 8,282,813 to Kaimori et al.; U.S. Pat. No. 8,702,921 to Frey et al.; and U.S. Pat. No. 9,080,957 to Ueno et al., all of which are incorporated by reference to the same extent as though fully replicated herein.

Table 1 below cites various articles that are incorp0oratd by reference to the same extent as though fully disclosed herein and identify various sensors that may be utilized in the sensor array 1602.

TABLE 1
Listing of Useful Sensor Types

| | |
|---|---|
| Biomarkers | https://www.chemyx.com/support/knowledge-base/applications/detecting- |
| | biomarkers-with-lab-on-a-chip/ |
| Glucose | Kim, N., Adhikari, K., Dhakal, R. et al. Rapid, Sensitive and Reusable |
| | Detection of Glucose by a Robust Radiofrequency Integrated Passive Device |
| | Biosensor Chip. Sci Rep 5, 7807 (2015). https://doi.org/10.1038/srep07807 |
| Temperature | Fathi, Yo. The MAX1464's On-Chip Temperature Sensor (2015) |
| O2 input, CO2 | Wencel, D. Sol-gel-derived optical oxygen, pH and dissolved carbon dioxide |
| output | sensors (2008). |

Atmospheric Detection of Contaminates

| | |
|---|---|
| Carbon | Texas Instruments Inc., Low-Power Carbon Monoxide Detector With BLE |
| Monoxide | and 10-Year Coin Cell Battery Life Reference Design (2016-2017) |
| VOC Detection | https://www.idt.com/us/en/products/sensor-products/gas-sensors |
| Humidity | https://www.ti.com/sensors/humidity-sensors/overview.html |

Biological threats

| | |
|---|---|
| Virus | CHINA Focus: Test chip for multiple respiratory viruses approved, |
| | Xinhua.net, Feb. 24, 2020. |
| COVID 19 | Researchers Develop Chip for Biomolecule Detection to Aid COVID-19 |
| | Testing, HospiMedica.com, Jun. 3, 2020. |
| Bacterial | Gomez-Sjoberg, R., Morisette, D., Bashir, R. Impedance Microbiology-on-a-Chip: |
| | Microfluidic Bioprocessor for Rapid Detection of Bacterial Metabolism. |

Spoors

| | |
|---|---|
| Anthrax | Cady, N., Stelick, S., Batt, C. PCR-based detection of Bacillus anthracis |
| | using an integrated microfluidic platform (2011) |
| Mold | Papireddy Vinayaka, P., van den Driesche, S., Blank, R., Tahir, M. W., |
| | Frodl, M., Lang, W., & Vellekoop, M. J. (2016). An Impedance-Based Mold |
| | Sensor with on-Chip Optical Reference. Sensors (Basel, |
| | Switzerland), 16(10), 1603. https://doi.org/10.3390/s16101603 |
| Pollen | https://amphasys.com/impedance-flow-cytometry/ |
| Weaponized | Scott A. Walper, Guillermo Lasarte Aragonés, Kim E. Sapsford, Carl W. |
| biologicals | Brown, Clare E. Rowland, Joyce C. Breger, and Igor L. Medintz |
| | ACS Sensors 2018 3 (10), 1894-2024 |
| | DOI: 10.1021/acssensors.8b00420 |

Other chemical threats

| | |
|---|---|
| Chemical | Krotz, D. Far from the laboratory, a microfluidic chip helps detect chemical |
| Weapons | and biological weapons, Berkeley Lab News Center, Sep. 27, 2002. |

FIG. 16 shows the face mask assembly 100, as described above, positioned on the head 1630 of a person wearing the face mask assembly. As shown in FIG. 16, a harness 116′ is connected to sliding locks 108, 110 and has respective upper and lower bands 1632, 1634 that separate to occupy different positions on the head 1630 such that an ear 1634 resides between the bands 1632, 1634 and is not contacted by the bands 1632, 1634.

### Purification Media

The purification media 500 can be any purification media known to the art. By way of example, the following patents of this paragraph, which are incorporated by reference to the same extent as though fully replicated herein, disclose fibrous materials for use in face masks: U.S. Pat. No. 6,732,733 to Brostrum et al, U.S. Pat. No. 5,706,804 to Baumann et al., U.S. Pat. No. 4,419,993 to Peterson, U.S. Reissue Pat. No. Re 28,102 to Mayhew, U.S. Pat. Nos. 5,472,481; 5,411,576 to Jones et at; and U.S. Pat. No. 5,908,598 to Rousseau et at The fibrous materials may contain additives to enhance filtration performance, such as the additives described in U.S. Pat. Nos. 5,025,052 and 5,099,026 to Crater et al., and may also have low levels of extractable hydrocarbons to improve performance; see, for example, International Publication No. WO 99/16945 by Rousseau et al. Fibrous webs also may be fabricated to have increased oily mist resistance using the techniques described in U.S. Pat. No. 4,874,399 to Reed et al., and in International Publication Nos. WO 99/16532 and WO 99/16533, both by Rousseau et M. Electric charge can be imparted to fibrous webs using techniques described in, for example, U.S. Pat. No. 5,496,507 to Angadjivand et al., U.S. Pat. No. 4,215,682 to Kubik et al., and U.S. Pat. No. 4,592,815 to Nakao.

The use of thermoplastic melt-fiber mats is preferred, according to the discussion below. These mats may utilize a thermoplastic felt that is heated to a temperature barely below the melting temperature of the thermoplastic. This may be, for example, a temperature that is one-half, one, two, three, four, five or even ten ° F. below the melting temperature (roughly 0.25 to 6° C.). The melting is sufficient to bind meso-fibers to the polymer felt and so also form a filtration matrix. Activated carbon meso fibers used in combination with the thermoplastic felt are particularly preferred.

The method disclosed herein may be further exemplified by the attached figures, which are broadly described below.

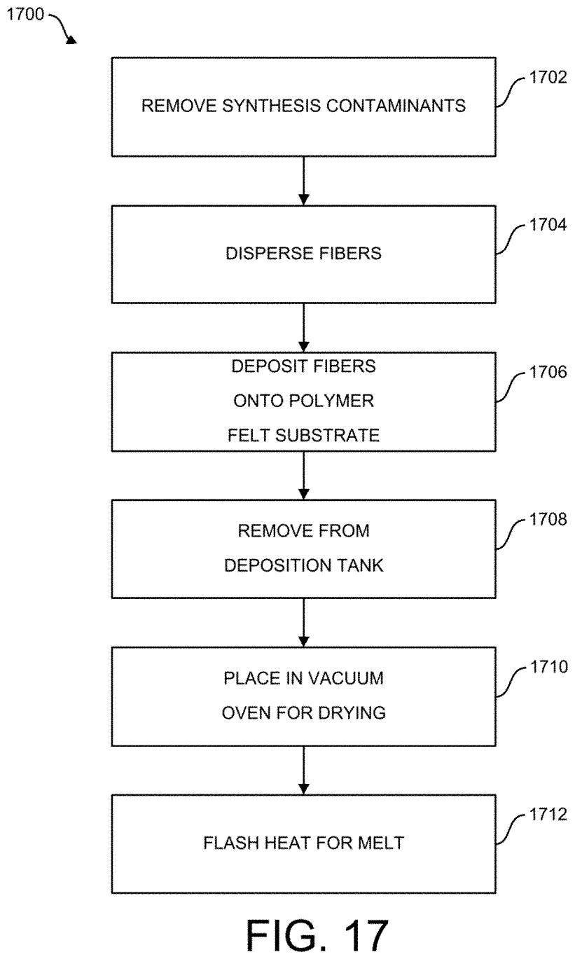

As shown in FIG. 17, a process 1700 according to one embodiment described below comprises a semi-continuous method of making the disclosed porous meso-structured composite material referred herein as the meso-composite using a modified paper-making type process.

To begin, remove 1702 synthesis contaminates from the meso-fibers with the previously disclosed nitric acid wash procedure. As a natural consequence of this, carboxyl groups will be created and covalently bonded to the accessible surface area of the meso-fibers with high density on any exposed edge of the meso-fibers. The carboxylation will assist in dispersion of the meso-fibers.

Next, disperse 1704 the meso-fibers, for example, by using ultra-sonication and mechanical mixing in a solvent solution comprising ethanol, water and optionally a binder. This suspension is referred to as the “meso-fiber suspension.”

The dispersed meso-fibers are deposited 1706 onto a polymer felt substrate. This is done, for example, by introducing the loaded meso-fiber suspension fluid into a deposition head box where the meso fiber-suspension encounters a porous polymer felt substrate. The felt substrate in turn may be supported by a flow restriction membrane and/or paper machine clothing or other permeable web support structure (not shown). This manner of deposition may be assisted by a differential pressure across the flow restriction membrane in an amount sufficient to obtain a substantially stable, interlocking, monolithic structure. In coincidence with the deposition process the deposition fluid is maintained in a substantially laminar flow regime. Optionally ultra-sonication may be applied to the suspension to maintain dispersion of the meso-fibers.

The newly formed meso-fiber/substrate structure and supporting structure are removed 1708 from the deposition tank and placed 1710 in a vacuum oven for drying. The drying may occur, for example, at a moderate temperature of between 50° C. and 80° C. under a partial vacuum to assist the drying and removal of the disclosed solvent from the meso-fiber/substrate structure.

The dried meso-fiber polymer composite media is then flash-heated 1712, preferably by electromagnetic radiation to achieve a temperature from 1° C. to 10° C. above the melting point of the polymer felt base. Such heating preferably occurs for a sufficient time to melt the polymer fiber at and only at the contact zone between the meso-fiber and the polymer fibers comprising the felt. Without being bound by theory the principle of surface energy minimization causes monolayers of polymer to flow onto the meso-fiber at the contact zone. Milliseconds later the polymer will solidify and fuse the meso-fiber to the polymer fiber. Carboxyl groups on the surface of the meso-fiber will further add adhesion strength between the disclosed meso-fiber and the polymer fiber. Preferably, this step is performed under vacuum conditions so that the atmospheric gases or water vapors do not form an unnecessary barrier to the welding dynamics.

The aforementioned process may be implemented either as a sequential batch process or as a reel-to-reel operation which is typically interrupted to reload rolls of the disclosed polymer felt. Alternatively, the polymer felt can be formed and fused on a reel-to-reel line upstream from the meso-fiber felt composite line. If a vacuum is used in a reel-to-reel operation then a multi-stage differential vacuum system must be built into the line after the drying stage. In order to establish the optimal conditions for the flash heat stage, performed under vacuum.

In such a system, the mechanical integrity of the deposition substrate should be sufficient to support the pressure differential by which the system operates, as well as be able to withstand any tension applied to the substrate to move it through the system. Optimally paper machine clothing is used as the support for the meso-fiber felt composite fabrication.

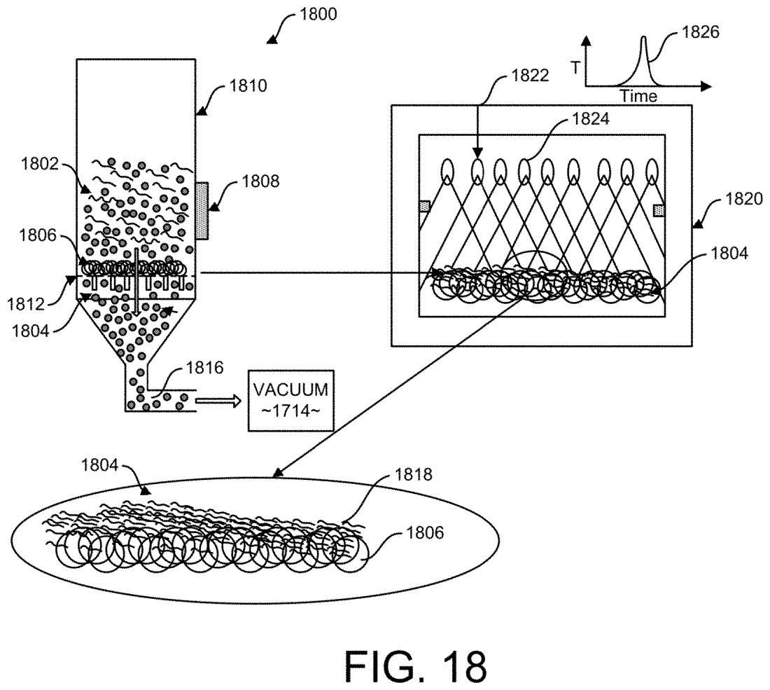

By way of example, FIG. 18 shows one embodiment of batch process equipment 1800 in which the meso-fiber suspension 1802 is deposited onto a substrate 1804 including a thermoplastic polymer felt 1806. The meso-fiber suspension 1802 contains, for example, as activated carbon meso-fibers and preferably activated carbon meso fibers. The substrate 1804 is porous and permeable only to the carrier fluid or solvent in the meso-fiber suspension 1802, but not for the suspended fibers in the carrier fluid, thus allowing the meso-fibers together with optional other materials, to deposit on the substrate 1804. One or more ultrasonication elements 1808 may attach to walls 1810 that define the deposition chamber and are used to maintain dispersion of the suspended materials in the meso-fiber suspension 1802. A non-linear flow restrictor 1812, such as a turbulator located slightly downstream or upstream of the substrate 1804, may be positioned proximate the substrate 1804, to enhance uniformity of deposition. A vacuum 1814 may be applied downstream of the substrate 1804 to induce a pressure differential across the substrate 1804 as an aid to deposition. Spent carrier fluid 1816 may be collected and optionally recycled.

After deposition, the substrate 1804, now laden with deposited meso-fibers 1818, is removed from within the walls 1810 of the deposition chamber and placed into a thermally controlled vacuum box 1820. Fast bright light emitting diodes (LEDs) 1822, 1824 are activated on a predetermined power profile 1826 to accelerate drying of the substrate 1804 and deposited meso fibers 1818 by removal of solvent that may continue to wet the same. The temperature controlled vacuum box 1820 is heated to a temperature that melts the thermoplastic felt 1804 and bind the meso-fibers top the polymer fibers. This may be, for example, a temperature that is one-half, one, two, three, four, five or even ten ° F. below the melting temperature (roughly 0.25 to 6° C.). The melting is sufficient to bind meso-fibers to the thermoplastic felt and so also form a filtration matrix that may be used as the purification 500 described above.

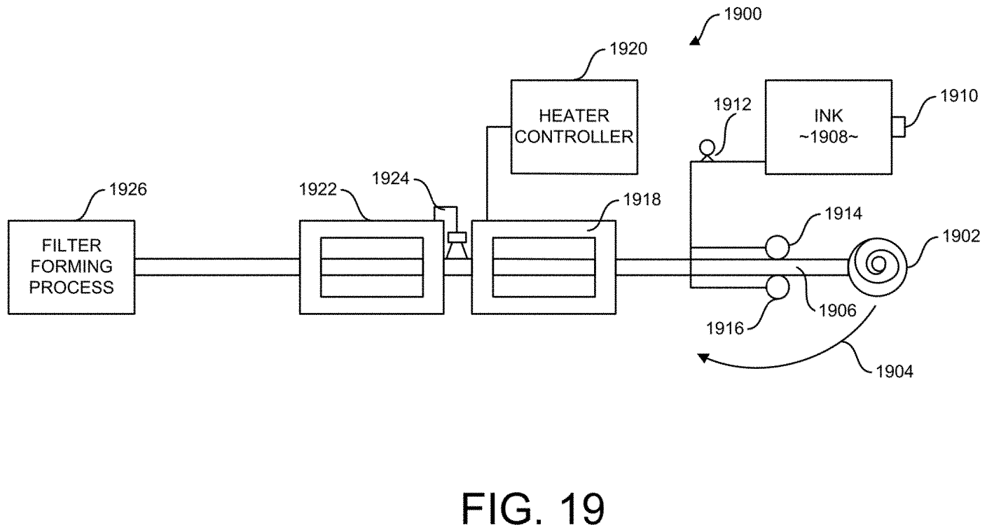

FIG. 19. shows one embodiment of web-based process equipment 1900 according to the presently disclosed instrumentalities. A roll 1902 of thermoplastic polymer felt is purchased on commercial order, and spooled to unwind in direction 1904 to form a moving web 1906. The polymer felt may be, more generally, a polymeric micro-fiber, for example, selected from synthetic polymers, biopolymers, proteins, cellulose, wool, cotton or any combination thereof. An ink reservoir 1908 contains, for example, a suspension of activated carbon meso-fibers in a solvent or dispersant mixed with a binder, such as a polymer precursor and a polymerization activating agent. The ink within reservoir 1908 may contain, for example, activated carbon meso fibers dispersed in water that is mixed with a binding agent for example, chitosan, DNA, RNA, synthetic polymers, biopolymers, or any combination thereof. Crossljinked chitosan is particularly preferred for use as the binding agent, in which case chitosan is a polymer precursor and formaldehyde acts as a polymerization agent that induces cross-linking of the chitosan. The amount of chitosan may be, for example, 1% by weight of the water. The amount of activated carbon meso-fibers is sufficient to remove undesirable material from a fluid, such removing bacterial, viruses, or volatile organic materials from air or water. This effective amount is suitably, by way of example, sufficient to provide a load of 2 g/m² activated carbon meso-fiber in a web 1906 having a thi8ckness of 1 mm.

In the case of a binding agent formed of chitosan and formaldehyde, the cross-linking reaction is preferably conducted using a stoichiometric excess of chitosan in order to consume substantially all of the formaldehyde. A sonicator 1910 maintains dispersal of the activated carbon meso-fibers. The ink may contain, for example, a suspension of activated carbon meso-fibers in a liquid including water formaldehyde mixed with a soluble form of chitosan. The amount of formaldehyde is provided to an amount that is stoichiometrically less than what is required to react with the chitosan in solution. The water is suitably mixed for example, with chitosan at a ratio of 100:1 by weight. The ink reservoir 1908 is cooled, for example, to 20° C. to limit the speed of this reaction.

One or more peristaltic pumps 1912 dispense precise quantities of the cooled, unreacted ink from reservoir 1908 to rollers 1914, 1916 which, in turn, apply the ink to the web 1906 from above and below as the web 1906 moves past the rollers 1914, 1916. The web 1906 enters a heating chamber 1918 that contains an internal atmosphere which is substantially saturated with water vapor and which is heated to a temperature that is sufficient to facilitate the reaction between the chitosan and the formaldehyde. This temperature may be suitably, for example, from 60° to 90° C. and is preferably from 80° to 85° C. The ensuing reaction binds the activated carbon meso-fibers in the ink to the web 1906. A controller 1920 operates on a sensor feedback circuit (not shown) that maintains the proper levels of temperature and humidity within the heating chamber 1918. A post-processing chamber 1922 optionally provides additional operations, such as spraying with water 1924 to remove residual formaldehyde and drying in chamber 1924.

The web 1906, now with dried, bound activated carbon meso-fiber, proceeds to purification-forming operations 1926 where, for example the web 1906 may be rolled and later cut to form the web 1906 into purifications 500 as described above or formed for placement in other purifications such as purifications of standard sizes and packaging for use in heating, ventilation and air-conditioning (HVAC) or filtration systems for the recirculation of air in aircraft or other vehicles. It will be appreciated that of certain metals or metal oxides may act as catalysts to remove volatile organic compounds (VOCs) when applied to activated carbon meso-fibers by processes described generally below. These catalysts may be, for example, oxides of tungsten, nickel, silver, and titanium.

The process equipment 1800 1900 may be utilized for repeat depositions to construct a multi-layered carbon media material wherein each layer may be of the same or different composition from other layers within the layered material. Further, each layer may be specifically designed to provide some desired behavior to the resulting multi-layer material. In addition, some of these layers may include layers not composed of meso-material and whose presence provides mechanical, electrical, and/or thermal properties or acts to set inter-membrane spacing for the carbon media layers.

### Purifications Made by the Process Equipment.

There is also provided in one aspect of the present disclosure a purification matrix made as described above for removing, disabling or destroying contaminants from a fluid, which may contain both liquid and gas contaminants.

Non-limiting examples of liquids that may be cleaned using the article described herein include air, water, foodstuffs, biological fluids, petroleum and its byproducts, non-petroleum fuels, medicines, organic and inorganic solvents, and the liquid forms of hydrogen, oxygen, nitrogen and carbon dioxide, as may be used for rocket propellants or in industrial applications.

Non-limiting examples of foodstuffs that can be treated with this article comprise animal by-products (such as eggs and milk), fruit juice, alcoholic and nonalcoholic beverages, natural and synthetic syrups, and natural and synthetic oils used in the cooking or food industry [such as olive oil, peanut oil, flower oils (sunflower, safflower), vegetable oil, or oils derived from animal sources (i.e. butter, lard)], or any combination thereof. As one example, sulfites are often added to wine to prevent discoloration and aid in preservation. However, sulfites raise health concerns and should be avoided. One aspect of the present disclosure could include the targeted removal of sulfites upon dispensing, benefiting the wine industry from the purification process described herein.

Biological fluids that may be decontaminated with the article described herein could be generally derived from an animal, human, plant, or comprise a culture/growth broth used in the processing of a biotechnology or pharmaceutical product. In one embodiment, the biological fluids which may be cleaned comprise blood (or blood components), serums, and milk. Biological reagents used in pharmaceutical products are often quite labile and difficult to sterilize by conventional techniques. Removal of small microorganisms (such as Mycoplasma and viruses) cannot be accomplished by conventional filtration. The inventive carbon media article may be used for viral removal without causing damage to the serum proteins often present and needed in biological reagents. In one embodiment, the physical and chemical properties of the carbon media can be controlled to enable removal of contaminants that are created during drug fabrication.

In another embodiment, the purification material may be used for the sterilization of petroleum products. A significant contamination problem is the latent growth of bacteria in petroleum or its derivatives during storage, which has been a problem particularly with aviation fuel. The presence of such bacteria can severely foul and eventually ruin the fuel. Accordingly, a major area of concern in the area of liquid purification is the cleaning bacteria from natural and/or synthetic petroleum products. Natural and/or synthetic petroleum and its byproducts include aviation, automotive, marine, locomotive, and rocket fuels, industrial and machine oils and lubricants, and heating oils and gases.

Another significant contaminant issue with petroleum products is high sulfur content and excessive levels of certain metals, a notable example being lead. Government regulations prohibit sulfur and lead concentrations in hydrocarbon fuels (used in internal combustion engines) in excess of specific amounts (MCL—maximum contamination level). Accordingly, there is a need for an article to remove specific chemical contaminants from petroleum without adding other unwanted constituents. In one embodiment, the article described herein can be used to remove sulfur and/or specific metals from hydrocarbon or other types of fuel, such as gases used in fuel cells.

As many of the foregoing contaminants may be dispersed in air, there is a need for a purification media for cleaning gases using a material made from the disclosed process. Accordingly, another aspect of the present disclosure includes a method of cleaning the air to remove any of the previously listed contaminants. Non-limiting examples of gases that may be cleaned using the article described herein include one or more gases chosen from the air or exhausts from vehicles, smoke stacks, chimneys, or cigarettes. When used to clean air, the article may take a flat form to provide a greater surface area for air flow. Such flat shapes provide the additional benefit of being able to be easily cut into appropriate shapes for various purification designs, such as those used in gas masks, as well as HVAC systems. The following gases that may be treated according to the present disclosure, such as scrubbed to clean the gas or remove them from exhaust, include argon, acetylene, nitrogen, nitrous oxide, helium, hydrogen, oxygen, ammonia, carbon monoxide, carbon dioxide, propane, butane, natural gas, ethylene, chlorine, or mixtures of any of the foregoing, such as air, nitrogen oxide, and gases used in diving applications, such as Helium/Oxygen mixtures.

Further, it should be noted that what might be identified as a contaminant in one fluid application may actually be a desired product in another. For example, the contaminant may contain precious metals or a beneficial pharmaceutical product. Therefore, in one embodiment, it may be beneficial to separate, retain and collect the contaminants rather than just removing and destroying them. The ability to “catch and release” desired contaminants, enabling the isolation of useful contaminants or certain reaction byproducts, may be accomplished by tuning the zeta potential and/or utilizing meso-electronic control of the carbon media article, as described in more detail below.

Applications for the articles described herein include home (e.g. domestic water and air filtration), recreational (environmental filtration), industrial (e.g. solvent reclamation, reactant purification), governmental (e.g. the Immune Building Project, military uses, waste remediation), and medical (e.g. operating rooms, clean air and face masks) locations.

While not necessary, the activated carbon meso-fiber media described herein can comprise activated carbon meso-fibers attached to each other, or to another material. The attachment and/or connection within the carbon media is a result of forces acting at the meso-scale, non-limiting examples of which are Van der Waals forces, covalent bonding, ionic bonding, geometric constraints, electrostatic, magnetic, electromagnetic, or Casimir forces or combinations thereof.

The present disclosure also relates to a method of purifying fluid by contacting contaminated fluid with the carbon media in the article described herein. In one embodiment, the method of purifying fluid comprises contacting the fluid with a carbon media, wherein the activated carbon meso-fibers are present in the carbon media in an amount sufficient to reduce the concentration of at least one contaminant in the fluid that comes into contact with the carbon media or the interaction zone created by the carbon media. As used herein “reduce the concentrations of at least one contaminant,” means a reduction of at least one contaminant to a level below that of the untreated fluid, such as below the maximum contamination levels (MCL) as defined by appropriate regulatory agencies or industrial requirements governing the quality standards of the particular fluid after being treated with the inventive article.

In one aspect, activated carbon meso-fibers whose morphology has been modified with a carbon dimer, alone or in patterns, can be used. For example, carbon dimers that have been inserted into two hexagonal bonds, creating two adjacent pentagons and heptagons in the chain link, can be used.

Activated carbon meso-fibers that comprise patterns of carbon dimers may also be used. Non-limiting examples of such activated carbon meso-fibers include: “bumpy” fibers, which have carbon dimers added symmetrically around the circumference of the fiber to create a stable bulge; “zipper” fibers, which have dimers added horizontally along the axial direction in every other hexagon, creating alternating single octagons and pairs of pentagons; and “multiple zipper” fibers, which have six axial “zippers” (described above) spaced by hexagonal rows around a fiber.

The phrase “exhibit a greater purification performance” means that the carbon media demonstrates either improvements to the structural integrity of the resultant material, its porosity, its porosity distribution, its electrical conductance, its resistance to fluid flow, geometric constraints, or any combination thereof that lead to an enhancement of contaminant removal. For example, greater purification performance could be due to improved and more efficient adsorption or absorption properties of the individual activated carbon meso-fibers. Further, the more defects there are in the activated carbon meso-fibers, the more sites exist for attaching chemical functional groups. In one embodiment, increasing the number of functional groups present in the carbon media should improve the performance of the resulting article.

### Treatment of Meso-Fibers

Unlike the previous discussion on the optional components that may be added to the carrier fluid, the following discussion relates to direct treatment of the activated carbon meso-fibers, which may be performed prior to dispersing the meso-fibers in the carrier fluid. It is noted however, that the disclosed method enables the treatment of the carrier fluid and the meso-fibers to be performed separately or in conjunction, depending on the desired result. For example, it is understood that the meso-fibers may be functionalized as described below, to aid in their dispersion in the carrier fluid, and that the carrier fluid may further comprise components that improve the integrity of the final product.

Thus, the activated carbon meso-fibers may also undergo chemical and/or physical treatments to alter their chemical and/or physical behavior prior to being added to the carrier fluid. These treatments are typically done to enable the resulting article to exhibit desired properties, such as a unique purification performance, in the sense defined above. Non-limiting examples of some unique purification properties are provided in the Examples of this disclosure.

In one embodiment, the activated carbon meso-fibers may be chemically or physically treated to achieve at least one of the following effects: remove contaminants, add defects, or attach functional groups to defect sites and/or meso-fiber surface.

Herein, “chemical or physical treatment” means treating with an acid, solvent or an oxidizer for a time sufficient to remove unwanted constituents, such as amorphous carbon, oxides or trace amounts of by-products resulting from the activated carbon meso-fiber fabrication process.

An example of the second type of chemical treatment is to expose the activated carbon meso-fibers to an oxidizer for a time sufficient to create a desired defect density on the surface of the activated carbon meso-fiber.

An example of the third type of the chemical treatment to attach specific functional groups that have a desired zeta potential (as defined in Johnson, P. R., Fundamentals of Fluid Filtration, 2nd Edition, 1998, Tall Oaks Publishing Inc., the definition of which is incorporated herein by reference). This will act to tune the zeta potential or the isoelectric point (pH where the zeta potential is zero) of the carbon allotrope sufficiently to remove a specific set of desired contaminants from a particular fluid.

The activated carbon meso-fibers described herein may also be treated to alter their properties, as well as the contaminants that may be removed from and/or modified within the fluid. For example, in one embodiment, the meso-fibers are chemically treated with an oxidizer, chosen from but not limited to a gas containing oxygen, nitric acid, sulfuric acid, hydrogen peroxide, potassium permanganate, and combinations thereof. Meso-fibers which have been treated with an oxidizer can provide unique properties, either in terms of fluid flow, dispersion of meso-fibers in the deposition fluid, or from a functionalization perspective (e.g., having the ability to be particularly functionalized).

Functionalization is generally performed by modifying the surface of activated carbon meso-fibers using chemical techniques, including wet chemistry or vapor, gas or plasma chemistry, and microwave assisted chemical techniques, and utilizing surface chemistry to bond materials to the surface of the meso-fibers. These methods are used to “activate” the meso-fiber, which is defined as breaking at least one C—C or C-heteroatom bond, thereby providing a surface for attaching a molecule or cluster thereto. In one embodiment, functionalized meso-fibers comprise chemical groups, such as carboxyl groups, attached to the surface, such as the outer sidewalls, of the meso-fiber. Further, the meso-fiber functionalization can occur through a multi-step procedure where functional groups are sequentially added to the meso-fiber to arrive at a specific, desired functionalized meso-fiber.

The functionalized activated carbon meso-fibers can comprise a non-uniform composition and/or density of functional groups including the type or species of functional groups across the surface of the meso-fibers. Similarly, the functionalized meso-fibers can comprise a substantially uniform gradient of functional groups across the surface of the meso-fibers. For example, there may exist, either down the length of one meso-fiber or within a collection of meso-fibers, many different functional group types (i.e. hydroxyl, carboxyl, amide, amine, polyamine and/or other chemical functional groups) and/or functionalization densities.

Further, other components of the carbon media, such as fibers and/or meso-particles, may also be functionalized with chemical groups, decorations or coatings or combinations thereof to change their zeta potential and/or cross-linking abilities and thereby improve the filtration performance of the carbon media.

A non-limiting example of performing a multi-step functionalization is one that allows the zeta potential of activated carbon meso-fibers to be controlled and improve their ability to remove viruses. The meso-fibers are refluxed in a mixture of acids. While not being bound by any theory, it is believed that such a process increases the number of defects on the surface of the meso-fiber, increasing carboxyl functional groups attached to the defect locations, and/or changes the zeta potential of the meso-fibers due to the negative charge of carboxyl functional groups in water.

Carboxyl functionalized meso-fibers may then refluxed in a solution of thionyl chloride in a nitrogen atmosphere. Without being held to any theory, it is believed that this acts to convert the previously attached carboxyl functional groups to acyl chloride functional groups. Subsequently, these acyl chloride functionalized meso-fibers are refluxed in as solution of ethylenediamine again in a nitrogen atmosphere. It is believed that this reacts with the amine groups on the end of the diamine with the acyl chloride functional group, thereby converting the acyl chloride functional group to a 2-aminoethylamide functional group by replacement of the chlorine atom with one amine group of the diamine. The termination of the meso-fiber functionalization with an amine group, will impart a positive charge to the meso-fiber in water, giving it a positive or less negative zeta potential. The foregoing would enable a filtration media device constructed with meso-fibers of this type to specifically target negatively charged contaminants (such as anions, certain molecules, and virus particles) for capture by Van der Waals and/or electrostatic forces, leading to their removal from the contaminant stream.

In another embodiment, activated carbon meso-fibers can also be used for high surface area molecular scaffolding either for functional groups comprised of organic and/or inorganic receptors or to provide structure and support for natural or bioengineered cells [including bacteria, meso-bacteria and extremophilic bacteria]. Examples of meso-bacteria, including images of meso-bacteria in carbonate sediments and rocks can be found in the following references, which are herein incorporated by reference. R. L. Folk, J. Sediment. Petrol. 63:990-999 (1993), R. H. Sillitoe, R. L. Folk and N. Saric, Science 272:1153-1155 (1996). The organic and/or inorganic receptors will selectively target the removal of specific contaminants from a fluid stream. The natural or bioengineered cells supported by the meso-fibers will consume, metabolize, neutralize, and/or bio-mineralize specific biologically-active contaminants. For example, there are specific microorganisms adhered to the meso-fibers that can reduce the toxicity of oil spills.

In another aspect of this disclosure, the activated carbon meso-fibers, or any subassembly thereof may be treated with radiation. The radiation may be chosen from but not limited to exposure from electromagnetic radiation and/or at least one particle chosen from electrons, radionuclides, ions, particles, clusters, molecules or any combination thereof. As previously described, the radiation should impinge upon the meso-fiber in an amount sufficient to 1) break at least one carbon-carbon or carbon-heteroatom bond; 2) perform cross-linking between meso-fiber-meso-fiber, meso-fiber to other carbon media constituent, or meso-fiber to substrate; 3) perform particle implantation, 4) improve the chemical treatment of the meso-fibers, or any combination thereof. Irradiation can lead to a differential dosage of the meso-fibers (for example due to differential penetration of the radiation) which causes non-uniform defect structure within the carbon media structure. This may be used to provide a variation of properties, via a variation of functional groups attached to the activated carbon meso-fibers.

In addition, activated carbon meso-fibers, according to the present disclosure, may be modified by coating or decorating with a material and/or one or many particles to assist in the removal of contaminants from fluids or increase other performance characteristics such as mechanical strength, bulk conductivity, or meso-mechanical characteristics. Unlike functionalized activated carbon meso-fibers, for example, coated or decorated activated carbon meso-fibers are covered with a layer of material and/or one or many particles which, unlike a functional group, is not necessarily chemically bonded to the meso-fiber, and which covers a surface area of the meso-fiber sufficient to improve the filtration performance of the carbon media.

Activated carbon meso-fibers used in the article described herein may also be doped with constituents to assist in the removal of contaminants from fluids. As used herein, a “doped” activated carbon meso-fiber refers to the presence of ions or atoms, other than carbon, into the crystal structure of the rolled sheets of hexagonal carbon. For example, doped activated carbon meso-fibers means at least one carbon in the hexagonal ring is replaced with a non-carbon atom.

In another embodiment, activated carbon meso-fibers as described herein could be decorated by a cluster or clusters of atoms or molecules. As used herein “decorated” refers to a partially coated meso-fiber. A “cluster” means at least two atoms or molecules attached by any chemical or physical bonding.

The clusters can exhibit properties of quantum dots resulting in photo-stable, color-tunable, meso-crystal with a wide absorption spectrum and a narrow emission peak. Clusters, including quantum dots, may be comprised of metals, nonmetals and combinations thereof. These attached clusters may be subsequently photo-activated to remove, disable and/or destroy contaminants. A quantum dot is a particle of matter so small that the addition or removal of an electron can be detected, and changes its properties in some useful way. In one embodiment, a quantum dot is a semiconductor crystal with a diameter of a few meso-meters, also called a meso-crystal, that because of its small size behaves like a potential well that confines electrons in three dimensions to a region of a few meso-meters.

The molecules or clusters may also include organic compounds containing at least one protein, including natural polymers composed of amino acids joined by peptide bonds, carbohydrates, polymers, aromatic or aliphatic alcohols, and nucleic or non-nucleic acids, such as RNA and DNA.

Non-limiting examples of the organic compound may comprise at least one chemical group chosen from carboxyls, amines, arenes, nitriles, amides, alkanes, alkenes, alkynes, alcohols, ethers, esters, aldehydes, ketones, polyamides, polyamphiphiles, diazonium salts, metal salts, pyrenyls, thiols, thioethers, sulfhydryls, silanes, and combinations thereof.

### Other Fibers Included in the Filter Media.

The carbon components of the purification media described herein may also comprise fibers which act to maintain the dispersion (or exfoliation) of these components during processing, and/or which may add mechanical integrity to the deposition substrate or the final product. Such fibers can be sacrificial (removed from the structure during further processing, such as by chemical or heat treatments) or can remain an integral part of the finished device. As used herein, the term “fiber” means an object of length L and diameter D such that L is greater than D, wherein D is the diameter of the circle in which the cross section of the fiber is inscribed. For example, the aspect ratio L/D (or shape factor) is chosen ranging, for example, from 2 to 109, such as from 5 to 107 and further such as from 5 to 106. Typically these fibers have a diameter ranging from 30 meso-meter to 100 meso-meters, such as from nm to 120 nm.

The fibers that may be used in the composition disclosed herein may be mineral or organic fibers of synthetic or natural origin. They may be short or long, individual or organized, for example, braided, and hollow or solid. They may have any shape, and may, for example, have a circular or polygonal (square, hexagonal or octagonal) cross section, depending on the intended specific application.

The fibers have a length ranging, for example, from 500 um to 10 m, such as from 800 um to 1.2 mm. Their cross section may be within a circle of diameter ranging, for example, from 80 nm to 120 nm.

The fibers can be those used in the manufacture of textiles as derived from bio-mineralization or bio-polymerization, such as silk fiber, cotton fiber, wool fiber, flax fiber, feather fibers, cellulose fiber extracted, for example, from wood, legumes or algae.

Medical fibers may also be used in the present disclosure. For instance, the resorbable synthetic fibers may include: those prepared from glycolic acid and caprolactone; resorbable synthetic fibers of the type which is a copolymer of lactic acid and of glycolic acid; and polyterephthalic ester fibers. Nonresorbable fibers such as stainless steel threads may be used.

In addition to activated carbon nano-fibers, the fibers may also contain:
(a) at least one polymeric material chosen from single or multi-component polymers such as nylon, acrylic, methacrylic, epoxy, silicone rubbers, synthetic rubbers, polypropylene, polyethylene, polyurethane, polystyrene, polycarbonates, aramids (i.e. Kevlar® and Nomee), polychloroprene, polybutylene terephthalate, poly-paraphylene terephtalamide, poly (p-phenylene terephtalamide), and polyester ester ketone, polyesters [e.g. poly(ethylene terephthalate), such as Dacron®], polytetrafluoroethylene (i.e. Teflon®), polyvinylchloride, polyvinyl acetate, viton fluoroelastomer, polymethyl methacrylate (i.e. Plexiglas), and polyacrylonitrile (i.e. Orlon®), and combinations thereof;
(b) at least one biological material or derivative thereof chosen from cotton, cellulose, wool, silk, and feathers, and combinations thereof; and
(c) at least one activated carbon meso-fiber chosen from 80 layers of graphene, 200 layer of graphene or multi-layer of activated carbon meso-fibers that have either a nested or non-nested morphology of meso-horns, meso-spirals, meso-springs, dendrites, trees, spider meso-fiber structures, meso-fiber Y-junctions, and bamboo morphology or multi-stranded helices;
(d) at least one metallic oxide or metallic hydroxide meso-wire. For example, a metal oxide meso-wire can be prepared by heating metal wires with oxygen in a reaction vessel to a temperature ranging from 230-1000° C. for a period ranging from 30 minutes to 2 hours. The meso-wires will grow by using macroscale wires made any metal previously mentioned as a feedstock. The resulting metallic oxide meso-wires can be in a size ranging from 1-100 meso-meters in diameter, such as 1-50 meso-meters in diameter, including 2-5 meso-meters in diameter. In one advantageous aspect of this process, the surface of the base wire is abraded to provide a roughened surface texture to enable better meso-fiber adhesion within media as well as enhance the purification performance of the article. These metal oxide or metal hydroxide meso-wires can also be obtained from commercial suppliers.

### Substrates Used in the Device.

One embodiment includes a support substrate for depositing the activated carbon meso-fibers using a differential pressure process, wherein the substrate is porous or permeable to the carrier fluid used to deposit the activated carbon meso-fibers. The porous support substrate may be in any form suitable for the shape of the resulting article, such as a block, tube (or cylinder), sheet or roll, and may comprise a material chosen from ceramic, carbon, metal, metal alloys, or plastic or combinations thereof. In one embodiment, the substrate comprises a woven or non-woven fibrous material.

Further, when the substrate takes the form of sheet, the substrate may be either a flat or planar sheet or in a pleated form. The pleated form being chosen to increase the surface area of the carbon media exposed to contaminated fluid, when used to purify contaminated fluids.

In one embodiment, the substrate is a roll of material on which the carbon media is deposited. In this process, the roll may be scrolled through a series of deposition and other processing stations in either a continuous or semi-continuous manner, as described above.

In another embodiment, wherein the carbon media is created by a rolled process, it may be used to wrap around a hollow, porous cylinder, block or other supporting structure to form the purification media.

In another embodiment, the porous tubular substrate comprises a carbon material, such as activated carbon (bulk or fiber), the outer surface of which is coated with the meso-fibers described herein.

In another embodiment, a collection of metal oxide/hydroxide meso-wires, made as described above, may also be used as a substrate for the deposition(s) of activated carbon meso-fibers using a differential pressure deposition process. The resulting meso-wire media may or may not be treated thermally, mechanically, or chemically to enhance structural integrity and/or improve the purification performance of the article. The chemical treatments may include the functionalizing, coating or decoration of the resultant media with chemical groups, metals, ceramics, plastics, or polymers. Further these chemical treatments may be done so that the media article chemically or physically reacts or interacts with contaminants to destroy, modify, immobilize, remove, or separate them.

In other embodiments, the porous support substrate used during the differential pressure deposition process may be either sacrificial or used only temporarily during deposition to form the carbon media in a method analogous to paper manufacturing.

### Other Manifestations of the Device

Another embodiment of the article comprises multiple carbon media layers, each of which may be specifically, and independently, tuned through its zeta potential or other means to remove a specific distribution of contaminants or to improve other performance characteristics of the article. The phrase, “other means” is intended to mean the tuning of specific properties of the media layer such as its porosity, the contaminant affinity [e.g. functionalization of media components, specific contaminant(s) receptors], or strength (e.g. binding or cross-linking agents used).

In another embodiment, the carbon media contains a binding agent (such as polyvinyl alcohol) that acts to improve the filtration performance of the article. Such a binding agent may be introduced into the suspension containing the meso-fibers and other media components prior to the formation of the purification media structure.

In another embodiment, the carbon media can be formed through a process of self assembly. “Self assembly” means that the purification media components arrange themselves into the final media structure. This is accomplished by controlling the electric, magnetic, chemical and geometric constraints through the choice of functional groups, surface charge distributions, the composition or properties of the dispersive agent, or any combination thereof. For example, adjusting the surface charge distribution of the carbon-based media components controls their electrical behavior, which in turn determines how they arrange into the structure of the assembled media. This self assembly may be in any form that leads to an enhanced structural framework within the media that improves the removal properties, porosity, electrical resistance, resistance to fluid flow, strength characteristics or combinations thereof.

Further, the above self assembly may be “directed” through the imposition of an external field. This applied field works in concert with the properties of any or all of the carbon media components and/or the fluid in which the components are suspended to guide their assembly into the resulting purification media. For example, a suspension containing some or all of the components of a carbon media may be subjected to electromagnetic stimulation during the formation of the carbon media to achieve a desired component alignment and/or weaving to enhance the fluid purification performance.

### Mechanisms of Action

1. Fluid Sterilization

Without wishing to be bound by any theory, it is believed the carbon media described herein forms a unique meso-scopic interaction zone that uses chemical and physical forces to first attract then to modify or separate microbes and other pathogens from the fluid stream. For example, it is believed that during the sterilization of a fluid, microorganisms come into contact with the carbon components of the media, causing focused forces to be applied to the microorganisms. These forces first attract, then either cause adherence and/or modification of cells. It is possible that this modification involves disrupting the cell membranes or causing internal cellular damage, thus disabling and/or destroying the microorganisms or their ability to reproduce. In this way, fluids can be effectively sterilized with respect to microorganisms. Common microorganisms are in the size range of 1-5 microns long and as such are at least 10 times larger than a meso-structure such as activated carbon meso-fibers. Known examples of these organisms include E. coli, Vibrio cholera, Salmonella typhi, Shigella dysenteriae, Cryptosporidium parvum, Giardia lamblia, Entamoeba histolytica, and many others. Examples of viruses transmitted through water include Polio, Hepatitis A, Rotavirus, Enteroviruses and many others. Examples of chemical agents include, but are not limited to, ions, heavy metals, pesticides, herbicides, organic and inorganic toxins, and microbial toxins (such as that causing botulism).

Due to the large size differences, forces on the meso-scopic scale can be applied that are orders of magnitude more intense than those based on micro- or macroscopic technologies. By analogy to the way that focused light gives the intensity to a laser, focused forces give the intensity to meso-scale attraction and/or destruction of microbes. Thus, mechanical and electrical forces that are on larger scales either too small to be effective or very energy-intensive, on the meso-scale can be used to effectively and efficiently remove or destroy microorganisms and viral particles.

Without being bound by theory mechanisms believed to be capable of adsorbing then destroying microorganisms viruses and spoors in this meso-regime can act independently or in concert with one another. Non-limiting examples of such mechanisms include:
Mechanical penetration and/or abrasion of the cell wall through focused forces;
Vibrational waves causing either external damage to the cell wall and transport channels and/or internal cellular damage to the DNA, RNA, proteins, organelles, and the like;
Bubble cavitations from shockwaves in the liquid around the activated carbon meso-fibers which damage the cell structure;
Electromagnetic, electrostatic and/or Van der Waals forces which capture and hold biological contaminants;
Disruption of hydrogen bonding in the vicinity of meso-structures via zeta action causing damage to cell walls and/or DNA;
Acidification of the environment around the meso-structure, due to specific meso-fiber functionalization that attract naturally occurring H+ ions in water, which damages cell walls and/or DNA.
Triggered release of the viral RNA from the viral shell, thus causing deactivation.
Fragmentation of a viral shell via the interaction between electric fields in the purification media and the protean shell.

Since the osmotic pressure within a typical microbial cell is higher than that of the surrounding fluid, assuming non-physiological conditions, even slight damage to the cell wall can cause total rupture as the contents of the cell flow from high to low pressure. Further, sufficient damage to the DNA of a viral or microbial cell can destroy at least one microorganism's ability to reproduce or infect host cells rendering it incapable of causing infection.

2. Meso-Electronic Fluid Purification

According to the present disclosure, another process of fluid purification is also based on the carbon media article. In this case, an electrostatic or electromagnetic field is imposed upon the media to control the purification of a fluid. Much like the behavior of electrostatic separation devices, the imposition of an electric potential across the media can remove contaminants on the meso-scale. Further, this process can be used in reverse to cleanse the purification article.

In addition, the entire carbon media can be stimulated with dynamic electromagnetic fields which, when properly adjusted, will excite media-wide vibrations. These vibrations could have both microorganism damaging effects or induce an ultrasonic self-cleaning effect. The utility of the inventive article, in this connection, is that advantage is taken of the high strength, high stiffness (large Young's modulus), high conductivity, and the piezoelectric property of the meso-fibers.

Additionally, for some applications, the imposition of a more generalized electromagnetic field can give fluid purification performance that goes beyond existing technologies. For example, in the case of two conducting carbon media layers, imposing an electric current generates a magnetic field between the layers. This field may be tuned to capture all charged particles from a fluid stream.

3. Liquid Desalination

According to the present disclosure, a process of liquid desalination is also based on the described carbon media article. One mechanism believed to be capable of desalinating liquid with the described media, is the imposition of a voltage differential between two or more carbon media membranes. In this case, one such media membrane carries a positive charge and the other membrane a negative charge. The applied potential causes cations to migrate toward the negatively charged membrane and anions to migrate toward the positively charged one. Due to the large surface area (1000 m2/gram) of these meso-fibers, the application a voltage differential across the media membrane creates a very high capacitance device, thereby creating an efficient, compact, reversible ionic separation zone (i.e. an ion trap).

A desalination unit could incorporate two or more parallel layers of supported conductive carbon media that are electrically isolated from each other. The two or more layers may be electrically charged in either a static or active mode. In static mode, for example, the media layers could be oppositely charged to create a salt trap between them. In an active mode device with four or more layers, for example, a four-phase signal would be applied to the multi-layer media structures such that the four legs of the signal are applied to four sequential media layers. This pattern is repeated every fourth media layer. In this way, the charge on each media layer and across the device indexes sequentially in time from positive to neutral to negative to neutral. Done sequentially in time would create, electronically, a moving virtual capacitor within the device which can cause the salt ions to migrate in a direction different than the flow of the water through the device. The concentrated salt water would accumulate at the terminus of the virtual capacitor and could be channeled out of a brine port on the device, while the fresh water would pass through the device.

In practice, due to the polarized nature of the water molecule, ions in a water solution have their charges shielded by a cloud of water molecules that surround them, which is described as the DeBye atmosphere. Because this cloud of water molecules is carried along with the ions as they move, it acts to increase the ions effective mass and ionic radius. Therefore, a higher frequency (relative to the frequency required to induce ion separation) AC signal can be imposed across the membrane layers in the desalination device. The purpose of this higher frequency signal is to disrupt the DeBye atmosphere shielding the ions in solution. As a result of shedding this water molecule shell, the ions appear smaller and less massive and can move with less resistance through the fluid. This aspect of the disclosure improves the efficiency of the desalination device.

Additionally, the desalination device described herein could be designed to take advantage of the biological removal characteristics of the carbon media structure, as discussed above, to purify the resulting fresh water.

4. Prevention of Bio-Films

According to one aspect of the present disclosure, surfaces susceptible to bio-film formation, due to the attachment and growth of contaminating microbes, can be coated with a layer of meso-material to prevent either the attachment or subsequent growth of undesirable elements, such as molds, bacteria. Non-limiting examples of such meso-materials include elements or compounds having antibacterial properties (such as iodine resin, silver, aluminum oxide, aluminum hydroxide, or triclosan) that are attached to the surface or located within the activated carbon meso-fiber or attached to any other carbon media component.

### Types of Contaminants Removable

Non-limiting examples of contaminants that can be removed from fluid using the disclosed article include, but are not limited to, the following biological agents: pathogenic microorganisms [such as viruses (e.g. smallpox, COVID-19, hepatitis), bacteria (e.g. anthrax, typhus, cholera), oocysts, spores (both natural and weaponized), molds, fungi, coliforms, and intestinal parasites], biological molecules (e.g. DNA, RNA), and other pathogens [such as prions and meso-bacteria (both natural and synthetic)].

“Prions” are defined as small infectious, proteinaceous particles which resist inactivation by procedures that modify nucleic acids and most other proteins. Both humans and animals are susceptible to prion diseases [such as Bovine Spongiform Encephalopathy (BSE or Mad Cow disease) in cows, or Creutzfeld-Jacob Disease (CJD) in humans].

“Meso-bacteria” are meso-scale bacteria, some of which have recently been postulated to cause biomineralization in both humans and animals. It has further been postulated that meso-bacteria may play a role in the formation of kidney stones, some forms of heart disease and Alzheimer's Disease. Further, meso-bacteria are also suspected of causing unwanted biomineralization and/or chemical reactions in some industrial processes.

Other non-limiting examples of contaminants that can be removed from fluid using the disclosed article include, but are not limited to noxious, hazardous or carcinogenic chemicals comprised of natural and synthetic organic molecules (such as toxins, endotoxins, proteins, enzymes, pesticides, and herbicides), inorganic contaminants (such as heavy metals, fertilizers, inorganic poisons) and ions (such as salt in seawater or charged airborne particles).

Applications of the cleaned fluid, specifically clean water, include potable water, irrigation, medical and industrial. For example, as a source of deionized water for industrial processes including, but not limited to, semiconductor manufacturing, metal plating, and general chemical industry and laboratory uses.

More specifically, the chemical compounds that may be removed from fluid using the article described herein are removal target atoms or molecules that include at least one atom or ion chosen from the following elements: antimony, arsenic, aluminum, selenium, hydrogen, lithium, boron, carbon, oxygen, calcium, magnesium, sulfur, chlorine, niobium, scandium, titanium, vanadium, chromium, manganese, iron, cobalt, nickel, copper, zinc, gallium, germanium, bromine, strontium, zirconium, yttrium, molybdenum, rhodium, palladium, iodine, silver, cadmium, indium, cesium, tin, barium, lanthanum, tantalum, beryllium, copper, fluoride, mercury, tungsten, iridium, hafnium, rhenium, osmium, platinum, gold, mercury, thallium, lead, bismuth, polonium, radon, radium, thorium, uranium, plutonium, radon and combinations thereof.

### Generalized Construction

Another aspect of the present disclosure relates to a method of making a carbon media material to be used in an article for removing contaminants from fluid, such as a carbon media material comprises functionalized activated carbon meso-fibers. Projected results as described below are based upon actual experiments rum using analogous graphene materials.

1. Preparation of Functionalized Activated Carbon Meso-Fibers

One process for preparing functionalized activated carbon meso-fibers generally comprises an initial sonication of commercially available meso-fibers in a solvent. Such meso-fibers include multi-wall carbon-structure meso-fiber powder made by any chemical process, such as Chemical Vapor Deposition (CVD) oven process that typically has a purity >95% by weight, and characteristic dimensions of 300 um 5 mm in length, such as 800 and 1.2 mm in diameter.

Therefore, subsequent to, or simultaneous with, sonication the activated carbon meso-fibers are treated in acid, chosen from but not limited to nitric, sulfuric, hydrochloric, and/or hydrofluoric acid or combination thereof. These acids can be used individually to wash the meso-fibers, or be used in various combinations. For example, in one embodiment, the meso-fibers are first washed in nitric acid and then washed in hydrofluoric acid. In another embodiment, the meso-fibers are washed in sulfuric acid after being washed in nitric acid.

The acid wash is performed to remove any contaminants, such as amorphous carbon, or catalyst particles and their supports which may interfere with the surface chemistry of the meso-fiber, and producing functional groups (such as carboxyl, for example) attached to the defect locations on the surface of the meso-fibers.

This functionalization also provides hydrophilicity to the activated carbon meso-fibers, which is thought to improve the filtration performance of the resulting article. The meso-fibers are then subjected to a final distilled water rinse, and suspension in an appropriate dispersant, such as distilled water, or an alcohol, such as ethanol or isopropanol. In one embodiment, sonication, stirring and heating is employed throughout this functionalization process to maintain adequate dispersion of the meso-fibers while cleaning.

2. Preparation of Metal Oxide Treated Fibers

In one embodiment, the process of making a carbon media for use in the described article comprises mixing the previously described functionalized meso-fibers with metal oxide (such as iron oxide) or metal hydroxide (such as iron hydroxide) treated (either coated or decorated) fibers as disclosed herein. The preparation of such metal oxide or metal hydroxide treated glass fibers may comprise mixing a metal oxide or metal hydroxide containing solution with commercially available glass fibers, such as fibers having a diameter ranging from 0.2 μm-5 μm.

In one embodiment, the process comprises stirring the glass fibers with a mixture of distilled water and colloidal metal oxide or metal hydroxide solution for a time sufficient to treat the glass fibers. The treated fibers may then be dried in an oven.

3. Preparation of Suspensions

The ingredients used to make the suspension comprise the functionalized activated carbon meso-fiber solution and the metal oxide or metal hydroxide treated fibers prepared in the previously mentioned processes. To prepare the component parts of the suspension, the functionalized meso-fibers are first dispersed in an appropriate medium, such as water or ethanol, by sonication. The metal oxide or hydroxide treated glass fibers are separately dispersed in a container, again in an appropriate medium, such as water or ethanol. These separate dispersions are then mixed to form a suspension of functionalized meso-fibers and metal oxide or metal hydroxide treated fibers.

In one embodiment, the structure of the final carbon media may comprise different layers of functionalized activated carbon meso-fibers and metal oxide or metal hydroxide treated glass fibers. These different layers are formed from distinct suspension made from different ratios of activated carbon meso-fibers and treated glass fiber.

4. Deposition of Media

The procedure for depositing the functionalized activated carbon meso-fiber/treated fiber mixture including, but not limited to, metal oxide or metal hydroxide coating of any of the fibers disclosed here in. For example, the media can be made from the meso-fiber/treated fiber mixture using a differential pressure deposition or direct assembly. In this embodiment, the deposition process uses differential pressure across the substrate to deposit the functionalized meso-fiber/metal treated fiber suspension onto a carbon block substrate. In this embodiment, the pressure difference applied across the substrate is such that the pressure is lower inside the substrate block. This differential pressure forces the fluid comprising the suspension to flow through the substrate, depositing meso-fiber/glass fiber mixture on the outer surface of the substrate, thereby forming the purification media.

5. Article Assembly

After the carbon media material is dried, the coated substrate may be covered with a porous protective paper and a coarse plastic netting to protect the media material. End caps are then attached and the edges of the carbon media sealed to prevent fluid circumventing the media. This assembly is then incorporated into an outer housing which is sealed to form the article for removing contaminants from a fluid.

### Methods for Determining Effectiveness

Using established microbiological techniques, described herein, it may be demonstrated the carbon media purifications are capable of removing more than 7 logs of a bacterial contaminant (E. coli) and more than 4 logs of a surrogate for viral agents (the MS 2 bacteriophage). These removal capacities exceed the requirements for bacterial removal and the recommended levels of viral removal specified by the US-EPA (Guidance Manual for Compliance with the Filtration and Disinfection Requirements for Public Water Systems Using Surface Water, U.S. Environmental Protection Agency, March 1991). Independent testing of the inventive article, will confirm that the article satisfies the basic standards for water purification in the United States.

Multiple tests are performed on samples made using the methods generally described above using bacteria, such as E. coli, and viruses, such as MS 2 bacteriophage. The MS 2 bacteriophage, which is commonly used as a surrogate in assessing a device's virus removal capabilities for drinking water and for air-purification, is a male specific, single stranded RNA virus, with a diameter of 0.025 um and an icosahedral shape. Its size and shape are similar to other waterborne viruses such as the polio and hepatitis viruses, although the MS 2 bacteriophage is not a human pathogen.

The protocol used for testing the removal of the E. coli bacteria and the MS 2 bacteriophage from water in the all of the following examples are consistent with and generally adhered to: (i) Standard Operating Procedure for MS 2 bacteriophage Propagation/Enumeration. Margolin, Aaron, 2001, University of New Hampshire, Durham, N.H. and (ii) Standard Methods for the Examination of Water and Wastewater, 20th Edition, Standard Methods, 1998, APHA, AWWA, WEF, Washington, D.C., which are herein incorporated by reference.

Using these methods described above, and as exemplified in the following examples, strong adherence forces between bacteria and activated carbon meso-fibers was activated carbon meso-tubes were observed. For example, the bacteria adhere to the activated carbon meso-fibers surface, especially when dispersed during sonication. It is believed that the same adherence of E. coli suspension occurs when it is passed through the disclosed carbon media of activated carbon meso-fibers.

In addition, evidence that the integrity of the bacterial cell, may be partially compromised upon interaction with the carbon media is observed. For example, electron microscopy of the bacteria in the presence of activated carbon meso-fibers described herein revealed images showing some apparent penetration of the bacterial shell/cell wall. After a prolonged period (24 hours) some disruption apparently resulted from a breech in the integrity of the cell wall, which, due to the difference in the osmotic pressure between the interior and exterior of the cell, leads to a catastrophic failure of the cell wall and the disintegration of the bacteria. However, this disruption of cell integrity was apparent immediately upon contact with the activated carbon meso-fibers, as observed by light microscopy in a phase microscope.

Further, tests will confirm the destruction of some bacteria, as evidenced by the presence of at least a small amount of free bacterial DNA and protein in the filtrate. However, most of the bacterial cells will remain intact immediately after contact with the meso-fibers. Although the inventive carbon media article will effectively remove bacteria from the effluent stream, the ability of the meso-fibers to kill bacterial cells has not yet been established, although it is a likely possibility.

Further, through other testing of the inventive article other contaminants, such as those previously described (including metals, salts, organic contaminants, endotoxins) may be removed from water and air.

The disclosed instrumentalities will be further clarified by the following non-limiting examples, which teach by way of example and not by limitation.

### Example 1: E. coli Interaction with Activated Carbon Meso-Fibers

The interaction of an E. coli bacterial culture with a suspension of activated carbon meso-fibers is investigated to determine the effectiveness of activated carbon meso-fibers to attach to and subsequently disable or destroy bacterial cells. Further, this study will provide insight into the mechanisms active in the inventive meso-purification article. The procedure compares an untreated sample containing bacterial cultures to a sample mixed with activated carbon meso-fibers. The comparisons are to be done under high magnification using both light and atomic force microscopy techniques.

Preparation of E. Coli Suspension

An E. coli suspension is made by using a sterile, biological loop (commercially available) to remove a loop full of the reconstituted stock [obtained from American Type Culture Collection (ATCC), stock culture ATCC #25922] which is streaked on a commercially available blood agar plate. This plate is then incubated for 12-18 hours at 36° C., removed from the incubator and examined for purity.

Using a sterile biological loop (commercially available) one loop full of the incubated culture is removed and placed in 10 ml of sterile commercially available Tryptic soy broth (Remel cat. No. 07228). The E. coli is then grown in the resulting trypticase-soy broth for 18 hours at 37° C., followed by centrifugation and suspension, to form a concentrated bacterial culture of approximately 5×109 colony forming units (cfu)/ml in pure water.

Functionalization of Activated Carbon Meso-Fibers with Nitric Acid

The activated carbon meso-fibers are treated with nitric acid solution to remove contaminants (such as amorphous carbon, or catalyst particles and their supports which may interfere with the surface chemistry of the meso-fiber), increase the number of crystalline defect sites in the meso-fibers and to attach carboxyl chemical group to these defect sites. This functionalization also provides a hydrophilic behavior to the activated carbon meso-fibers.

The treatment is performed by mixing 250 mg of purified meso-fibers in a total volume of 35 ml of concentrated nitric acid in a centrifuge tube, shaking well and sonicating in a Cole Parmer 8851 Sonicator at full power for 10 minutes in 50° C. water bath. The nitric acid/activated carbon meso-fiber mixture are then centrifuged at 2,500 rpm until the supernatant is clear (6-10 minutes) and then the supernatant was decanted. The nitric acid treatment is repeated, but with 20 minutes of sonication. The nitric acid treated activated carbon meso-fibers are then water washed by suspending them in 35 ml total volume distilled water, sonicating (as above) for 10 min, centrifuging (as above), then decanting the supernatant. This water wash is repeated until the pH is at least 5.5 (˜3-4 times), sonicating for 5 min each time.

Preparation of Test Solutions

The E. coli suspension, prepared as outlined above, is then divided into two equal parts. The untreated solution (Test Solution #1) is prepared by diluting one of the divided E. coli suspensions with distilled water to attain an E. coli concentration of ^{˜}2×109 cfu/ml (2:5 dilution). The other solution (Test Solution #2) was prepared by adding 25 mg of functionalized meso-fibers to the other divided E. coli suspension. This solution is then diluted with distilled water to achieve the same concentration of E. coli as in Test Solution #1. This dilution results in a concentration of activated carbon meso-fibers in Test Solution #2 of 625 ppm.

Both Test Solutions #1 and #2 are simultaneously sonicated with a Branson-2510 Sonicator for 3 min. These Test Solutions are then centrifuged in a commercially available centrifuge at 2500 rpm for 2 minutes to form pellets, and the supernatant decanted leaving 1 ml of supernatant behind. The pellets of Test Solutions #1 and #2 are then used to make two samples (#1 and #2) described below.

Preparation of Sample #1: Activated Carbon Meso-Fiber Free

Sample #1 is prepared by placing a drop of the test solution free of activated carbon meso-fibers (Test Solution #1) on a commercially available glass microscope slide (American Scientific Products, Micro Slides, plain, Cat. M6145, size 75×25 mm that is cleaned with sulfuric acid and rinsed with distilled water) and refrigerated at 4° C. for 19 hours. After refrigeration, atomic force microscopy (AFM) analysis was performed (without fixation) using a Veeco Dimension 3100 Scanning Probe System in tapping mode to investigate the sample.

Sample #1 is also thermally fixed (by brief exposure to an open flame) and then stained (with Gram Crystal Violet dye) followed by a water wash. Light microscopy is performed using an Olympus light microscope at 1000× magnification and under immersion oil. Digital images were made with an Olympus DP10 CCD.

Preparation of Sample #2: Treated Activated Carbon Meso-Fiber

Sample #2 is prepared by placing (and smearing) a drop of the activated carbon meso-fiber/E. coli test solution (Test Solution #2) on a glass microscope slide as described above. The sample is thermally fixed, stained, and light microscopy is conducted as for Sample #1 above. Sample #2 is then placed in a refrigerator at 4° C. for 19 hours, after which time it is removed and AFM analysis (as described above) is conducted as for Sample #1. Sample #2 is returned to the refrigerator for an additional 24 hours, after which time light microscopy is again conducted.

Results of Microscopic Analyses

Sample #1 (suspension of bacteria without activated carbon meso-fibers) shows E. coli bacterial cells uniformly distributed over the entire surface of the slide. The image further shows that the bacteria had well-defined edges, suggesting that the bacteria cells were intact. No changes in their shape are found after 2 days stored in a dry state in the refrigerator.

The results for samples from the activated carbon meso-fiber treated test solution (Sample #2) will demonstrate bacteria clumped on the activated carbon meso-fibers. The majority of the meso-fibers are removed when the excess stain was washed from the slide. Bacteria concentration is observed at boundaries of the activated carbon meso-fibers.

There are numerous individual bacterial cells present over the entire slide for the sample without activated carbon meso-fibers (Sample #1) bacterial cells are absent from most of the slide for the sample with activated carbon meso-fibers (Sample #2). Any bacteria that are present in the latter case are tightly packed around the activated carbon meso-fibers, indicating that the activated carbon meso-fibers are capturing and holding the bacteria.

Sample #1 demonstrated E. coli closely packed together. The bacterial cells of normal cells will have sharp boundaries. The decrease in size and packing density of bacteria is seen in the AFM image of sample #1 before heat treatment and optical image of this sample after heat treatment.

Sample #2 shows some cells in the vicinity of the meso-fibers, with the boundary of the E. coli cell walls being diffused and/or damaged. After mixing with the meso-fibers, some of the E. coli cells disintegrate beyond the point of recognition. The presence of some diffused E. coli fragments may also be seen in the vicinity of the meso-fibers.

On sonication of E. coli and functionalized activated carbon meso-fibers in distilled water, the two components agglomerate. This is thought to be due to electrostatic and Van Der Waals forces which act at the meso-scale. To the limit of detection, it is observed that all bacteria in suspension are in contact with the meso-fibers, and adhered. There are no longer free E. coli cells in Solution #2. This illustrates the ability of the dispersed activated carbon meso-fibers to strongly attach to and immobilize bacteria.

The disintegration of the E. coli cells, when noted, appears after the cells come into intimate contact with the meso-fibers. As a result, these bacteria cells appear to lose their sharp cell boundaries and their internal contents spill out from the cell.

In the cells affected, the beginning of this process is noted after 3 hours, and after 22 hours the internal contents spread so far that it is difficult to distinguish the shape of the cell.

A highly motile bacterium, Pseudomonas fluorescens, grown for 12 hours in nutrient broth (from Difco Laboratory) at room temperature, is mixed with a solution of activated carbon meso-fibers. Viewed under a dark field microscope, the motile bacteria are observed to swim near and get pulled into the aggregated activated carbon meso-fibers and become firmly attached to the exposed activated carbon meso-fiber fibers. Within 5 minutes of contact, the entire surface of the activated carbon meso-fiber aggregate is covered with hundreds of intact bacteria, which are obviously firmly attached since they appear to struggle, but are unable to leave. These bacteria lose all motility and become completely rigid within 30 seconds of initial contact with activated carbon meso-fiber fibers. This indicates the capacity of the finely dispersed activated carbon meso-fibers fibers to rapidly attach to and immobilize large numbers of bacteria. This will confirm the basis for the effectiveness of activated carbon meso-fiber purification media in removing microorganisms.

### Example 2: Construction of Cylindrical Purification Article

a. Iron Hydroxide Treated Glass Fiber Preparation

A solution of 23.5 liters of distilled water and 9.62 ml of 10N sodium hydroxide (NaOH) is made and stirred for 1 hour. A quantity of 16.66 grams of Ferric Chloride (FeCl3.6H2O) is added and stirred until a final pH of ^{˜}2.2 was reached (^{˜}24 hours). To this solution, 200 grams of glass fibers of size 100-500 nm in diameter and 300-500 μm in length (Johns-Mansville) are added and stirring is continued until solution is clear of iron (^{˜}3 hours). The solution is diluted with distilled water to obtain a glass fiber concentration of 10 grams/liter.

b. Preparation of Depositional Suspension

A suspension is prepared using a solution of functionalized activated carbon meso-fibers and iron hydroxide treated glass fibers previously prepared as described above. To prepare the component parts of the suspension, 5 g of the functionalized activated carbon meso-fibers (carboxylated through the nitric acid wash procedure described in Example #1) are suspended in 1 liter of water and placed in a room temperature water bath in a Cole Parmer 8851 Sonicator and sonicated at full power for 20 minutes. Four liters of distilled water are added to the sonicated, functionalized activated carbon meso-fibers/water mixture to yield a concentration of 1 mg functionalized activated carbon meso-fibers per 1 ml water. Approximately 100 ml of Fe decorated glass fiber solution is placed in a separate container and diluted to 1 liter with distilled water. This mixture is blended in a commercial blender for 5 minutes.

To mix the first depositional suspension, 600 ml of the suspended functionalized activated carbon meso-fibers (described above) are added to 960 ml of the glass fiber solution (5:8 CNT/glass ratio by weight). This mixture is diluted to 4 liters by adding a quantity sufficient amount of distilled water, and sonicated with a Branson model 900B probe Sonicator for 10 minutes on full power.

c. Example Deposition of Carbon Media

The structure of the final carbon media is achieved by depositing a layer of the functionalized activated carbon meso-fibers/iron hydroxide coated glass fiber mixture onto a carbon block substrate.

The procedure for depositing the functionalized activated carbon meso-fiber/iron hydroxide coated or decorated glass mixture is described as follows. A purification assembly is made by loading a cylindrical carbon block onto a perforated mandrel. The deposition chamber is filled with the activated carbon meso-fiber/glass fiber suspension (5:8 ratio). The purification assembly is connected to vacuum tubing leading to a Franklin Electronics Varian TriScroll vacuum pump and then was submerged in the filled deposition chamber. The vacuum pump attached to the purification assembly is turned on and the entire suspension is drawn through the carbon purification substrate under vacuum, depositing a carbon media on its outer surface. After deposition, the deposited purification assembly is removed from the deposition chamber, and remains connected to the vacuum pump while the deposited carbon media purification assembly is dried under vacuum for 1-2 hours in a drying oven set at 50° C. within a nitrogen atmosphere.

The fully assembled purification article is comprised of a central carbon purification core coated with the functionalized activated carbon meso-fiber carbon media and covered by a porous protective paper held in place with cylindrical plastic netting. This cartridge is capped and the edges of the carbon media sealed to prevent fluid circumventing the carbon media and placed into an outer housing to create the final product.

### Effectiveness of Cylindrical Purification Article

As a fluid purification test of the cylindrical form of the inventive article on water contaminated is conducted with an E. coli bacterial culture [obtained from American Type Culture Collection (ATCC)].

A bacterial assay is conducted by challenging the carbon media, made in accordance with the present example (Example 2), with a challenge fluid of reconstituted E. coli stock culture ATCC #25922. This challenge fluid is made by using a sterile biological loop (commercially available) to remove a loop full of the reconstituted stock and streaking it on a commercially available blood agar plate. This plate is then incubated for 12-18 hours at 36° C. The culture is then removed from the incubator and examined for purity.

Using a sterile biological loop (commercially available) one loop full of the incubated culture is removed and placed in 10 ml of sterile commercially available Tryptic soy broth (Remel cat. No. 07228). E. coli is then grown in the resulting trypticase-soy broth 18 hours at 37° C. to form a culture of approximately 1×109 colony forming units (cfu)/ml. A 1 ml sample of this stock culture is added to 100 ml of water to be used for the challenge test, thereby diluting the concentration to approximately 1×107 cfu/ml. The resulting challenge water is then passed through the Cylindrical Purification Article.

The test is performed in accordance with the “Standard Methods for the Examination of Water and Waste Water” cited above. Results of tests following the protocols described above establish consistent removal of E. coli bacteria greater than 6 logs (>99.99995%) to greater than 7 logs (>99.999995%) when the challenge fluid was passed through the inventive carbon media. These test results established removal rates which exceed EPA potable water standards (referenced above) for removal of bacteria from water. The EPA standards dictate 6 logs removal (>99.99995%) of E. coli bacteria to achieve potable water. Improved purification by greater log removals of E. coli bacteria may be achieved in such tests, by passing a solution of known bacterial concentration (i.e. challenging) the carbon media with higher concentrations of E. coli bacteria challenge suspension, made as described above. Such tests with higher concentrations will confirm removal rates of greater than 7 log (>99.999995%). Independent tests of the carbon media, using the test procedures described in this example, establish this material as a barrier to E. coli bacteria. Further, independent laboratory tests results show more than 6 logs of removal of different test bacteria (Klebsiella terrigena and Brevindomonas), confirming that the material is a general barrier to bacteria.

### Example 3: Fabrication of a Flat Purification Article

Analogously to Example 2, a flat carbon media is made from commercially available purified activated carbon meso-fibers and a non-woven, fused, polypropylene fabric substrate. To begin, 100 mg of functionalized activated carbon meso-fibers (carboxylated through a nitric acid wash as described in Example #1) are then added to 400 ml of commercially available neat isopropanol and sonicated in a “Branson 900B Ultrasonicator” at 80% power until the activated carbon meso-fibers were well dispersed (about 10 minutes). The mixture is further diluted by adding 2 liters isopropanol such that the total volume of the resulting mixture was 2.4 liters. This diluted mixture is sonicated for an additional 10 minutes.

Next, 800 mg of a commercially available 200 nm diameter glass meso-fiber is homogenized in a commercially available blender at full power for 10 minutes in 500 ml of the commercially available neat isopropanol. The homogenized mixture is then diluted by adding an additional 1 liter of commercially available neat isopropanol.

The mixtures of activated carbon meso-fibers and glass meso-fibers are combined and then quantity sufficient (Q.S.) amounts of isopropanol was added to obtain 4 liters. This 4 liter solution is then sonicated with a “Branson 900B Ultrasonicator” at 80% power for 15 minutes, which causes the activated carbon meso-fiber meso-material to uniformly disperse.

The entire 4 liter solution is then drawn through a commercially available 5 micron, non-woven, fused activated carbon fabric under a differential pressure of 1 atmosphere to deposit the activated carbon meso-fiber/treated glass fiber carbon media. The resulting carbon media is removed from the fabricator and allowed to dry in an oven at 50° C. for 2 hrs.

The resulting flat, square carbon media/substrate membrane is glued, using an NSF compliant hot-melt adhesive, into one side of a flat housing. This half of the housing is then mated and glued to its companion to seal.

Test of Effectiveness of Flat Purification Article

a. Water Contaminated with E. coli—Chemical Analysis

The following example describes the hypothetical results of a chemical analysis of filtrate from an E. coli challenge test, performed as described in Example 2, on the Flat Carbon Media Purification Article made in accordance with present example. Projected results are based upon actual experiments rum using graphene meso-fibers. This example is intended to provide evidence for the destruction of E. coli bacteria passing through the inventive carbon media. This evidence of partial destruction of the contaminant (E. coli bacteria) is established by the presence of bacterial DNA and proteins in the challenge filtrate.

A challenge test is run following the same procedures as in Example 2, except that the composition of the challenge solution was ^{˜}1×108 cfu/ml of E. coli. A total of 100 ml (total ^{˜}1×1010 cfu) of this challenge solution is drawn through the carbon media/substrate material using a differential pressure of ^{˜}0.25 psi. A control filtrate was obtained by passing the E. coli challenge filtrate through a commercially available 0.45 micron Millipore filter. The test challenge filtrate was not concentrated. The resulting filtrates, of the control and the challenge, are then analyzed with a commercially available spectra-photometer to determine the presence of protein and DNA. However, the analysis of the filtrate with a commercially available spectra-photometer reveals about 40 μg/ml of DNA and 0.5 mg/ml of protein. Concentrations of protein and DNA at these levels in non-concentrated challenge filtrate are 6 times higher than the control test material obtained by filtration through a Millipore filter. These concentrations confirm the destruction of at least some portion of the added E. coli by the carbon media.

b. Water Contaminated with MS-2 Bacteriophage Virus

The Flat Purification Article, made are described above, is tested with water contaminated by MS-2 bacteriophage virus using the procedure described above and in the “Standard Operating Procedure for MS-2 Bacteriophage Propagation/Enumeration, Margolin, Aaron, 2001, An EPA Reference Protocol.” MS-2 bacteriophage virus is commonly used in assessing treatment capabilities of membranes designed for treating drinking water (NSF 1998). The pressurized challenges for this example are performed with 100 ml challenge solutions using the protocols described above. The MS-2 challenge materials are prepared in accordance with those steps enumerated above.

In this test, eighty (80) membranes comprised of the activated carbon meso-fiber meso-structured material made in accordance with the present example are challenged. The challenge material used is water contaminated with MS-2 bacteriophage virus to the concentration of approximately 5×106 plaque forming unit (pfu)/ml.

Of the 80 units tested, 50 units achieved MS-2 removal of 5 logs (99.999%) or greater than 5 logs (>99.9995%). The remaining 30 units demonstrate 4 logs (99.99%) or greater than 4 logs (>99.995%) removal of MS-2. While EPA standards recommend 4 logs removal of MS-2 Bacteriophage to achieve potable water, it is believed that better sensitivity (higher log removal) may be achieved by challenging with higher log challenges of MS-2. Improved purification by greater log removals of MS-2 Bacteriophage may be achieved in such tests, by challenging the activated carbon meso-fiber carbon media, made in accordance with the present example with higher concentrations of MS-2 Bacteriophage challenge suspension, made as set forth above. Independent tests of the carbon media article, made in accordance with the present example (Example 3), will establish this material as a barrier to MS-2 Bacteriophage.

c. Water Contaminated with Arsenic (as)

The Flat Purification Article, made in accordance with the present example (Example 3), with water contaminated with arsenic. In this test, a 100 ml water solution containing ^{˜}150 ppb (parts per billion) arsenic is passed through the carbon media made in accordance with the present example (Example 3). A sample of the arsenic treated water is analyzed according to the EPA Method #SM 183113B. The analysis of the challenge filtrate confirms a reduction of the arsenic level by 86%±5%; after passing the challenge arsenic treated water, once through the inventive carbon media material.

d. Aircraft Fuel Contaminated with Bacteria

The Flat Purification Article, made in accordance with the present example, is tested for contaminated jet fuel. A sample of contaminated jet fuel (JP8) was obtained from a 33,000 gallon storage tank located at the United States Air Force Research facility at the Wright Patterson Air Force base. After collection, the sample is cultured on trypticase-soy agar and found to contain three types of bacteria: two Bacillus species and one Micrococcus species. The sample is separated in two containers of 2 liters each. Both containers present two distinct layers, jet fuel on top and water on the bottom. Container A contains a heavy contaminated growth layer at the interface between the water and the fuel. Container B only shows slight contamination. The challenge test bacteria are obtained from the interface of the fuel and water from Container B.

After being homogenized, which is accomplished by shaking the challenge test fuel/water/bacteria vigorously for 1 minute, 200 ml of the fuel/water/bacteria challenge mixture is passed one time, using 1.5 psi differential pressure, through the activated carbon meso-fiber, meso-structured material, made in accordance with the present example.

The fuel/water/bacteria challenge filtrate sample is allowed to separate into its fuel-water components, and four test samples were obtained from each component. Each test sample is plated on agar. Samples are then incubated to analyze bacteria growth at 37° C. and samples are incubated at room temperature to analyze mold growth. No bacteria or mold culture growth is observed on the challenge filtrate test plates after incubating the samples for 24 and 48 hours. The control samples present vigorous colonies of bacteria and mold growth after incubation at 24 and 48 hours. The results are projected to confirm that the carbon media, made in accordance with the present example (Example 3), is a barrier to bacteria in fuel for it accomplished removal of bacteria and mold from the fuel beyond the limits of detection with testing protocols.

### Example 4: Flat Purification Article Using a Multistep Functionalization

A flat carbon media device is made from commercially available, purified, activated carbon meso-fibers and a non-woven, fused, 0.5 oz/yd2 carbon tissue paper substrate. The construction of this device utilizes a process of self-[assembly of the carbon media, as defined above. Specific electropositive and electronegative functional components are used to enable this self assembly. The activated carbon meso-fibers were functionalized with amine groups which causes them to be electropositive (i.e. positive zeta potential) when dispersed in water. The glass fibers are decorated with iron hydroxide clusters that causes them to be electronegative when dispersed in water. When the two suspensions are combined, the meso-fibers wrap around the glass fibers due to electrical forces.

To begin, 20 g of activated carbon meso-fibers are refluxed with 400 ml of 60% 36N sulfuric acid and 40% 15.8N nitric acid at 110° C. for 30 minutes. This procedure is known to add carboxyl functional groups to the activated carbon meso-fibers. These carboxyl functionalized meso-fibers are filtered, washed in distilled water and then dried in an oven at 100° C. The dry meso-fibers are then suspended in 500 ml thionyl chloride and sonicated 20 hours at 60° C. The thionyl chloride is distilled off and the activated carbon meso-fiber sample is dehydrated using a vacuum pump. The dehydrated meso-fibers are suspended in 500 ml of ethylenediamine and sonicated for 20 hours at 60° C. in a nitrogen atmosphere. The ethylenediamine is distilled off and the sample washed with 0.1 M hydrochloric acid, filtered and rinsed repeatedly with distilled water until a neutral pH is reached. The rinsed amine functionalized activated carbon meso-fibers are then dried in an oven at 100° C. for 24 hours.

A mixture of 360 mg of amine functionalized activated carbon meso-fibers and 960 mg of treated glass fibers are combined and then a quantity sufficient (Q. S.) amount of distilled water is added to obtain 4 liters. This 4 liter solution was then sonicated with a “Branson 900B Ultrasonicator” at 80% power for 15 minutes, which causes the activated carbon meso-fiber/glass fiber meso-material to uniformly disperse.

The entire 4 liter solution is then drawn through a commercially available, non-woven, fused, 0.5 oz/yd2 carbon tissue under a differential pressure of ^{˜}1 atmosphere to deposit the self-assembled, activated carbon meso-fiber/treated glass fiber carbon media. The resulting carbon media is removed from the fabricator and allowed to dry in an oven at 50° C. for 2 hours.

The resulting flat, square carbon media/substrate membrane is glued, using an NSF compliant hot-melt adhesive, into one side of a flat housing. This half of the housing is then mated and glued to its companion to seal.

### Test of Effectiveness of Flat Purification Article

The flat purification device constructed in the present example (Example #4) using the amine functionalized activated carbon meso-fibers and iron hydroxide decorated glass fibers is tested for biological removal as in described in the Tests of Effectiveness for Example #3 [test a) E. coli and b) MS-2 bacteriophage]. These tests demonstrate that the self-assembled carbon media article achieves a removal capability for bacteria and virus of over 8 logs and 7 logs, respectively.

### Example 5: Fluid Desalination

A 64 layer, flat carbon media device is made from: commercially available purified, functionalized activated carbon meso-fibers; glass fibers measuring 100-500 nm in diameter and 300-500 μm in length; a solution of 0.0125% by weight of polyvinyl alcohol with a molecular weight of 20,000 g in distilled water; 1.5 oz/yard cellulose filter paper as an insulator; a non-woven, fused, 0.5 oz/yard conductive carbon tissue paper substrate; silver-imbedded conductive and insulating epoxies; a plastic, non-conductive housing; and a power supply to supply 1.5V DC across each neighboring pair of conducting carbon media layers.

To begin, 25 mg of functionalized meso-fibers (carboxylated through a nitric acid wash procedure as described in Example #1) and 50 mg of glass fiber (described above) are suspended in 4 liters of distilled water containing a 0.0125% concentration of polyvinyl alcohol as listed above. The suspension is stirred for 3 minutes using an IKA UltraTurrax T18 immersion blender at speed 3.

This activated carbon meso-fiber/glass fiber suspension is deposited on a 5″×5″ area of a 5.5″×5.5″ sheet of 0.5 oz/yard carbon tissue paper using differential pressure of ^{˜}1 psi. Four 2″ diameter discs are cut from this 5″×5″ carbon media sheet, thereby completing 4 layers of the 64 layer, 2″ diameter device (32 of the 64 layers are conductive, the others are insulating).

An electrical lead is attached to each conductive carbon media layer using a silver-filled conductive epoxy. All conductive carbon media layers are sandwiched between insulating layers and these “sandwiches” are then stacked with the electrical leads being equally spaced azimuthally (i.e. rotated ^{˜}11.25° from the leads on the layer above and below). The electrical leads are bundled and routed through the plastic housing wall to the power supply and the entire assembly is sealed.

A static retention test is performed by flowing 1 liter of a 1‰ saline solution (1‰=1 g salt/1000 g water) through the device with no electrical charge or stimulation imposed. The filtrate was tested for salt content and it is found to have lost ^{˜}13 mg of salt. Therefore the device in static mode (i.e. no electronic stimulation) reduces the salinity by ^{˜}1.3%. This reduction amounts to 0.42 grams of salt removed per gram of activated carbon meso-fibers in the device.

A dynamic retention test is performed, wherein a differential DC voltage of 4.0 mV was applied to each of 16 neighboring pairs of conductive carbon media layers (i.e. even numbered carbon media layers are positively charged and odd numbered layers are negatively charged). A saline challenge solution of 1 g of sodium chloride dissolved in 1000 ml of distilled water (1‰ salinity) was used to test the efficacy of the device. In one pass through the device, 1.6% of the salt is removed. This removal rate is equivalent to 0.52 g of salt per g of activated carbon meso-fibers. This represents a 23% increase in salt removal over the static device, showing that even a very weak voltage enhanced the removal of salt ions from a water solution, thereby demonstrating the meso-electric removal effect. Further enhancement of the salt removal will be achieved as the DC voltages are increased and AC signals, which disrupt the DeBye atmosphere, are imposed.

### Example 6: Air Membrane

A flat air purification matrix constructed using functionalized activated carbon meso-fibers (carboxylated through the nitric acid wash as described in Example #1). The procedure suspends 25 mg of these functionalized meso-fibers in 25 ml of distilled water and sonicated for 10 minutes in a Branson Model 900B Sonicator in a water bath at room temperature. This solution is then diluted to 4 liters with distilled water and polyvinyl alcohol is added so that a concentration of 0.125% polyvinyl alcohol by weight is achieved. The suspension is then mixed for 3 minutes at speed setting 3 with an UltraTurrax T18 Basic immersion blender. The carbon media is created by deposition on a 5″×5″ area of a 5.25″×5.25″ square piece of porous, polymeric substrate using a differential pressure filtration process with a differential pressure of ^{˜}1 psi.

### Test of Effectiveness of Air Membrane Article

Biological removal testing is performed on the membrane to determine its effectiveness. Two 2.5″ discs were cut from the square membrane and are mounted between two flat metal rings of 2″ ID, 2.5″ OD. One disc is used to measure the pressure drop versus flow speed curves for the membrane article device, while the other is used for biological removal testing. The bio-removal testing is done by mounting the filter disc in a 2″ ID cylindrical wind tunnel which is capable of testing the capture efficiency of bacterial spores of Bacillus subtilis, a widely accepted surrogate for biological agents but not a human pathogen, making it safe for laboratory testing.

The testing entails releasing the bacterial spores upstream of the filter disc through an aerosolizer and capturing the fraction that passed through the purification media in a fluid-filled, all-glass impinger at the downstream end of the testing apparatus. A controlled set of experiments are performed to estimate the spore retention of the testing apparatus. In this biological testing, we project to achieve over 6 logs of removal of Bacillus subtilis spores. Further, we are able to determine that removal of biological agents is independent of the removal of non-biological particles and of the purification media's resistance to air flow.

### Example 7: Reel-to-Reel Manufacturing Process

The example is related to a process for making a meso-structured material according to the present disclosure. This example describes the pre-processing of each component material, their combination in the carrier fluid, and the deposition of the carrier fluid onto and through a moving substrate. Post treatment of the deposited meso-structured material and testing of the performance of the meso-structured material is also described.

### Pre-Processing of Component Materials

a. Activated Carbon Meso-Fibers

The activated carbon meso-fibers were treated with nitric acid solution to remove contaminants, such as amorphous carbon, which may interfere with the surface chemistry of the meso-fiber. This treatment step also was performed to increase the number of crystalline defect sites in the meso-fibers and to attach carboxyl chemical group to these defect sites. A 75 g batch of functionalized meso-fibers was created from several smaller batches. In these smaller batches, the treatment was performed by mixing 20 mg of purified meso-fibers suspended in 600 ml of distilled water with a total volume of 450 ml of 70% concentrated nitric acid.

This mixture was poured into a glass beaker which was then placed in a 70° C. sonication bath and stirred for 30 minutes. The nitric acid/activated carbon meso-fiber mixture was then poured into a Buchner funnel and the acid was drawn off of the activated carbon meso-fibers using vacuum filtration. These nitric acid treated activated carbon meso-fibers were then water washed 3-4 times with distilled water (roughly 4 liters total volume was used) until the pH was at about 5.5. They were then suspended in 75 liters of reverse osmosis treated water. The functionalized activated carbon meso-fiber mixture was processed through a Microfluidics high-pressure disperser using a 75 μm diameter dispersing head and 10 kPsi pressure drop to break up meso-fiber agglomerations.

b. Glass Fibers

A mixture containing 600 g of glass fibers was prepared from Johns-Manville Code 90 glass fibers suspended in 120 liters of reverse osmosis treated water and stirred for 60 minutes. This fiber mixture was passed through a Silverson Model 200L High Shear In-Line Mixer operating at 75 Hz with a general purpose disintegrating head. These glass fibers were coated with a thin iron hydroxide coating by adding to the mixture a 1 liter solution containing 220 g of Fe(NO3)3.9H2O. This mixture was stirred well until the color equalized and the pH was recorded. This mixture was then covered and allowed to age for 60 hours under constant stirring.

A 4 liter solution of 0.50N sodium hydroxide was prepared and added automatically at a rate of 2 ml/min to the iron/glass mixture using a Millipore Waters Model 520 pump for 24 hours. This titration was continued until a pH of 3.95±0.05 was reached. At this point the titrated solution was allowed to age for another 2 days to complete the Iron (III) Hydroxide coating procedure. The final pH value after the additional aging period was 4.60±0.05.

### Preparation of Suspension and Dispersion

After pre-processing, the component materials were combined as follows. A suspension was prepared using the functionalized activated carbon meso-fibers and iron hydroxide treated glass fibers mixtures prepared as described above. To mix the depositional suspension, 75 liters of the 1 gram/liter functionalized activated carbon meso-fiber suspension was added to 120 liters of the 5 gram/liter glass fiber solution and passed through a Greerco model AEHNXU X0022 in-line, dual-head, high-shear mixer to obtain 195 liters of a 1:8 meso-fiber to glass ratio (by weight) suspension.

### Deposition of Activated Carbon Meso-Fibers of Activated Carbon Meso-Tubes and Glass Fibers

Sonication was used to achieve and/or maintain adequate dispersion of the fiber/activated carbon meso-fiber suspension on its path to the depositional head-box of the reel-to-reel meso-material production equipment. The combined activated carbon meso-fiber/glass fiber suspension prepared as described above was pumped through a static, Archimedes-screw type mixing element and then sequentially through Advanced Sonics 4 kW and 20 kW, 16/20 kHz dual frequency, in-line Sonicators at a flow rate of 12 gal/min using a Seepex model 12F-90 L/4 CUS progressive cavity pump.

After being prepared, the fiber/activated carbon meso-fiber suspension was supplied to the head box of a 18″ wide Fordrinier type paper making machine running at 20 feet/minute. This suspension was deposited upon a substrate composed of Blue Thunder Novatech-1000 substrate material and the resulting material was covered with Reemay 2014 spunbond as protection for subsequent machine and manual handling and rolling. No post-treatment was performed.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained according to the present disclosure.

Other embodiments will be apparent to those skilled in the art from consideration of the specification and practices disclosed herein. It is intended that the specification and examples be considered as exemplary only, with a true scope and spirit of the invention being indicated by the following claims.

We claim:

1. A face mask comprising:
a main frame element that is permeable to air;
a bulbous flexible face guard constructed to conform with sufficient flexion providing means for sealing engagement between a rearward wall thereof against the contours of a human face to prevent leakage of air therebetween; and
a front cover element that is permeable to air,
at least permeable purification media element residing between the main frame and the front cover,
the purification media being permeable to air and including conductive activated carbon meso-fibers supported by a network of micro fibers, and a binding agent connecting the conductive activated carbon meso-fibers and the a network of micro polymeric fibers,
the conductive activated carbon meso-fibers being present in an effective amount for the purification-removal of unwanted constituents from air,

2. The face mask of claim 1, further comprising electronic enhancements to the purification media.

3. The face mask of claim 2 in which the electronic enhancements include at least one member selected from the group consisting of circuitry establishing a potential difference across the filter media; static charging due to airflow through a purification media comprising conductive activated carbon nano-fibers acting as electron donors and insulative micro-fiber material micro-fiber material acting as electron acceptor material, piezoelectric devices, capacitive devices, photo-voltaic cells, batteries, sensors, wires, digital circuitry, analog circuitry or any combination thereof.

4. The face mask of claim 1 in which the main frame and the front cover are formed as ridged porous elements made of at least one material selected from the group consisting of thermal set polymer material, epoxy, metal, ceramic, and wood.

5. The face mask of claim 1 wherein the network of micro-fibers includes at least one material selected from the group consisting of synthetic polymers, biopolymers, proteins, cellulose, wool, and cotton.

6. The face mask of claim 1 wherein the binding agent includes at least one material selected from the group consisting of chitosan, cross-linked chitosan, DNA, RNA, synthetic polymers, biopolymers, or any combination thereof.

7. The face mask of claim 1 in which the said face guard is made of at least one material selected from the group consisting of a soft biocompatible: silicon polymer, and bio-polymer.

8. The face mask of claim 1, further comprising a harness for retaining the face mask on the head of a wearer.

9. The face mask of claim 1, further comprising a layer of bio-compatible adhesive located between the said flexible face guard and the intended face for retaining the face mask on the head of a wearer.

10. The face mask of claim 8 in which the harness attaches at sliding locks connected to the main frame, the sliding locks being formed in two pieces and are coupled by a bayonet latch mechanism.

11. The face mask of claim 1 in which the purification media contains at least one metal oxide catalyst for the removal of volatile organic compounds from air.

12. The face mask of claim 1 in which the purification media is functionalized to facilitate the removal of biological pathogens from air.

13. A purification media element comprising:
polymeric microfibers
conductive activated carbon meso-fibers,
and a binding agent coupling the polymeric microfibers and the conductive activated carbon meso-fibers,
the purification media being permeable to air flow,
the activated carbon meso-fibers being present in effective amounts for the purification-removal of unwanted constituents from air.

14. The purification media of claim 13 in which the purification media is contains at least one metal oxide catalyst for the removal of volatile organic compounds from the fluid.

15. The purification media of claim 13 in which the purification media is functionalized to facilitate the removal of viruses from air.

16. The purification media of claim 13 in which the purification media is functionalized to facilitate the removal of bacteria from air.

17. The purification media of claim 13 incorporated as a purification element to a face mask.

18. The purification media of claim 13 further comprising a metal oxide catalyst for the removal of volatile organic compounds from air.

19. The purification media of claim 13 in which the activated carbon meso-fibers are functionalized to facilitate removal of pathogens from air.

20. The purification media of claim 13 formed in a standard size for use as a purification in at least one of a filtration system, a heating system, ventilation system, an air-conditioning system, and a system for recirculation of air in a vehicle.
